(19) United States
(12) Patent Application Publication
(10) Pub. No.: US 2022/0348653 A1
(43) Pub. Date: Nov. 3, 2022

(54) Quantitative Spatial Profiling for LAG-3 Antagonist Therapy

(71) Applicant: Bristol-Myers Squibb Company, Princeton, NJ (US)

(72) Inventors: Cyrus HEDVAT, Princeton, NJ (US); Robin EDWARDS, Princeton, NJ (US); George C. LEE, Princeton, NJ (US); Vipual Atulkumar BAXI, Princeton, NJ (US)

(73) Assignee: Bristol-Myers Squibb Company, Princeton, NJ (US)

(21) Appl. No.: 17/762,576

(22) PCT Filed: Sep. 22, 2020
(86) PCT No.: PCT/US2020/052021

§ 371 Date: Mar. 22, 2022

Related U.S. Application Data
(60) Provisional application No. 62/903,887, filed on Sep. 22, 2019.

(51) Int. Cl. C07K 16/28; A61P 35/04; A61K 9/00

(52) U.S. Cl. CPC C07K 16/2803; A61P 35/04; A61K 9/0019; A61K 2039/505

(57) ABSTRACT

The disclosure provides LAG-3 antagonists and methods comprising the same for treating a cancer in a subject based on a LAG-3 density score and/or a LAG-3 proportion score in a tumor sample from the subject. The disclosure also provides methods of identifying a subject responsive to a LAG-3 antagonist therapy.

### CROSS REFERENCE TO RELATED APPLICATIONS

This PCT application claims the priority benefit of U.S. Provisional Application No. 62/903,887, filed Sep. 22, 2019, which is incorporated herein by reference in its entirety.

### FIELD OF DISCLOSURE

The present disclosure provides LAG-3 antagonist therapies for treating cancer in a subject based on quantitative spatial profiling of LAG-3 and major histocompatibility complex class II (MHC II) in a tumor sample from the subject.

### BACKGROUND OF THE DISCLOSURE

LAG-3 (CD223) is a type I transmembrane protein that is expressed on the cell surface of activated CD4+ and CD8+ T cells and subsets of NK and dendritic cells (Triebel F, et al., J. Exp. Med. 1990; 171:1393-1405; Workman C J, et al., J. Immunol. 2009; 182(4):1885-1891). LAG-3 is closely related to CD4, which is a co-receptor for T helper cell activation. Both molecules have four extracellular Ig-like domains and bind to MHC II. In contrast to CD4, LAG-3 is only expressed on the cell surface of activated T cells, and its cleavage from the cell surface terminates LAG-3 signaling. LAG-3 can also be found as a soluble protein but its function is unknown.

T cells that are continuously exposed to antigen become progressively inactivated through a process termed “exhaustion.” Exhausted T cells are characterized by the expression of T cell negative regulatory receptors, predominantly Cytotoxic T-Lymphocyte Antigen-4 (CTLA-4), Programmed Cell Death 1 (PD-1), and LAG-3, whose action is to limit the cell's ability to proliferate, produce cytokines, and kill target cells and/or to increase Treg activity. However, the timing and sequence of expression of these molecules in the development and recurrence of tumors have not been fully characterized.

The promise of the emerging field of personalized medicine is that advances in pharmacogenomics will increasingly be used to tailor therapeutics to defined subpopulations, and ultimately, individual patients in order to enhance efficacy and minimize adverse effects. However, unlike the clinical development of small molecule agents that target discrete activating mutations found in select cancer populations, a particular challenge in cancer immunotherapy has been the identification of predictive biomarkers to enable patient selection and guide on-treatment management.

A need exists for biomarkers and targeted therapeutic strategies that identify patients who are more likely to respond to a particular anti-cancer agent and, thus, improve the clinical outcome for patients diagnosed with cancer.

### SUMMARY OF THE DISCLOSURE

The present disclosure is directed to a method of treating a cancer in a human subject in need thereof comprising administering a lymphocyte activation gene-3 (LAG-3) antagonist to the subject, wherein the subject is identified as having (i) a high LAG-3 density (LAG-3-D) score, (ii) a high LAG-3 proportion (LAG-3-P) score, or (iii) both a high LAG-3-D score and a high LAG-3-P score in a tumor sample obtained from the subject, wherein the LAG-3-D score is determined by measuring a density of T cells expressing LAG-3 in proximity to one or more tumor cells expressing major histocompatibility complex class II (MEW II) in the tumor sample, and wherein the LAG-3-P score is determined by measuring a proportion of T cells expressing LAG-3 in proximity to one or more tumor cells expressing MHC II in the tumor sample.

The present disclosure is directed to a method of treating a cancer in a human subject in need thereof comprising (a) identifying a subject having (i) a high LAG-3-D score, (ii) a high LAG-3-P score, or (iii) both a high LAG-3-D score and a high LAG-3-P score in a tumor sample obtained from the subject and (b) administering a LAG-3 antagonist to the subject, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample.

The present disclosure is directed to a method of identifying a human subject afflicted with a cancer suitable for a LAG-3 antagonist therapy, comprising computing (i) a LAG-3-D score, (ii) a LAG-3-P score, or (iii) both a LAG-3-D score and a LAG-3-P score in a tumor sample obtained from a subject in need of a LAG-3 antagonist therapy, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample. In some aspects, the subject exhibits a high LAG-3-D score, a high LAG-3-P score, or both a high LAG-3-D score and a high LAG-3-P score. In some aspects, the method further comprises administering a LAG-3 antagonist to the subject.

The present disclosure is directed to a LAG-3 antagonist for treating a cancer in a human subject in need thereof, wherein the subject is identified as having (i) a high LAG-3-D score, (ii) a high LAG-3-P score, or (iii) both a high LAG-3-D score and a high LAG-3-P score of a tumor sample obtained from the subject, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample.

The present disclosure is directed to a LAG-3 antagonist for identifying a subject afflicted with a cancer suitable for a LAG-3 antagonist therapy, wherein (i) a LAG-3-D score, (ii) a LAG-3-P score, or (iii) both a LAG-3-D score and a LAG-3-P score in a tumor sample obtained from the subject is computed, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample. In some aspects, the subject exhibits a high LAG-3-D score, a high LAG-3-P score, or both a high LAG-3-D score and a high LAG-3-P score.

In some aspects, the LAG-3-D score is calculated as (i) the number of the T cells expressing LAG-3 in proximity to the tumor cells expressing MHC II divided by (ii) the tumor area (mm²) of the tumor sample.

In some aspects, the LAG-3-P score is calculated as (i) the number of the T cells expressing LAG-3 in proximity to the tumor cell expressing MHC II divided by (ii) the total number of T cells expressing LAG-3 in the tumor sample.

In some aspects, the proximity is between the LAG-3 and the MHC class II and/or between the LAG-3 and a tumor antigen expressed on the tumor cells.

In some aspects, the proximity is equal to or less than about 50 μm, equal to or less than about 45 equal to or less than about 40 equal to or less than about 35 or equal to or less than about 30 μm.

In some aspects, the proximity is equal to or less than about 30 μm.

In some aspects, the tumor sample comprises one or more tumor sections derived from a tumor tissue biopsy or a tumor tissue resection. In some aspects, the one or more tumor sections comprise a formalin-fixed, paraffin-embedded tumor tissue or a fresh-frozen tumor tissue. In some aspects, the one or more tumor sections comprise serially sectioned tumor sections. In some aspects, the one or more tumor sections are stained by immunohistochemistry (IHC). In some aspects, the one or more tumor sections comprise one tumor section, two tumor sections, three tumor sections, four tumor sections, five tumor sections, six tumor sections, seven tumor sections, eight tumor sections, nine tumor sections, ten tumor sections, 11 tumor sections, 12 tumor sections, 13 tumor sections, 14 tumor sections, 15 tumor sections, 16 tumor sections, 17 tumor sections, 18 tumor sections, 19 tumor sections, 20 tumor sections, 21 tumor sections, 22 tumor sections, 23 tumor sections, 24 tumor sections, 25 tumor sections, 26 tumor sections, 27 tumor sections, 28 tumor sections, 29 tumor sections, or 30 tumor sections. In some aspects, one tumor section of the tumor sample is stained for the LAG-3 and the MHC II. In some aspects, the tumor section is further stained for a tumor antigen, e.g., Pan cytokeratin (CK). In some aspects, the tumor sample comprises a first tumor section stained for the LAG-3, a second tumor section stained for the MHC II, and a third tumor section stained for a tumor antigen. In some aspects, the first tumor section, the second tumor section, and the third tumor section are serially sectioned from the tumor sample.

In some aspects, the high LAG-3-D score is at least about 5 cells/mm², at least about 10 cells/mm², at least about 15 cells/mm², at least about 20 cells/mm², at least about 25 cells/mm², at least about 30 cells/mm², at least about 35 cells/mm², at least about 40 cells/mm², at least about 45 cells/mm², at least about 50 cells/mm², at least about 55 cells/mm², at least about 60 cells/mm², at least about 65 cells/mm², at least about 70 cells/mm², at least about 75 cells/mm², at least about 80 cells/mm², at least about 85 cells/mm², at least about 90 cells/mm², at least about 95 cells/mm², or at least about 100 cell s/mm².

In some aspects, the high LAG-3-P score is at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In some aspects, the subject exhibits improved overall survival or progression free survival compared to a non-responder (a subject with a low LAG-3 D score, a low LAG-3-P score, or both).

In some aspects, any of the above methods or LAG-3 antagonist for uses further comprise measuring a tumor mutational burden (TMB) status.

In some aspects, the subject exhibits a high TMB.

In some aspects, any of the above methods or LAG-3 antagonist for uses further comprise measuring membranous PD-L1 expression in the tumor. In some aspects, the tumor is PD-L1 positive.

In some aspects, the tumor is LAG-3 positive.

In some aspects, the LAG-3 antagonist is a soluble LAG-3 polypeptide. In some aspects, the soluble LAG-3 polypeptide is a fusion polypeptide. In some aspects, the soluble LAG-3 polypeptide comprises a ligand binding fragment of the LAG-3 extracellular domain. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha).

In some aspects, the LAG-3 antagonist is an anti-LAG-3 antibody.

In some aspects, the anti-LAG-3 antibody is a full-length antibody. In some aspects, the anti-LAG-3 antibody is a monoclonal, chimeric, humanized, human, or multispecific antibody. In some aspects, the multispecific antibody is a dual-affinity re-targeting antibody (DART), a DVD-Ig, or bispecific antibody.

In some aspects, the anti-LAG-3 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-LAG-3 antibody cross-competes with BMS-986016 (relatlimab) for binding to human LAG-3.

In some aspects, the anti-LAG-3 antibody binds to the same epitope as BMS-986016 (relatlimab).

In some aspects, the anti-LAG-3 antibody is BMS-986016 (relatlimab), LAG-525 (IMP-701, ieramilimab), MK-4280 (28G-10), REGN3767 (fianlimab), TSR-033, TSR-075, Sym022, FS-118, IMP731 (H5L7BW), GSK2831781, humanized BAP050, aLAG3(0414), aLAG3(0416), XmAb22841, MGD013, BI754111, P 13B02-30, AVA-017, 25F7, AGEN1746, or comprises an antigen binding portion thereof.

In some aspects, the LAG-3 antagonist is administered at a flat dose.

In some aspects, the LAG-3 antagonist is administered at a weight-based dose.

In some aspects, any of the above doses is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, any of the above methods or LAG-3 antagonist for uses further comprise administering to the subject an additional therapeutic agent.

In some aspects, the additional therapeutic agent comprises an anti-cancer agent.

In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor comprises sorafenib, lenvatinib, regorafenib, cabozantinib, sunitinib, brivanib, linifanib, erlotinib, pemigatinib, everolimus, gefitinib, imatinib, lapatinib, nilotinib, pazopanib, temsirolimus, or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof.

In some aspects, the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof.

In some aspects, the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the checkpoint inhibitor comprises a PD-1 pathway inhibitor. In some aspects, the PD-1 pathway inhibitor is an anti-PD-1 antibody and/or an anti-PD-L1 antibody.

In some aspects, the PD-1 pathway inhibitor is an anti-PD-1 antibody. In some aspects, the anti-PD-1 antibody is a full-length antibody. In some aspects, the anti-PD-1 antibody is a monoclonal, chimeric, humanized, human, or multispecific antibody. In some aspects, the multispecific antibody is a dual-affinity re-targeting antibody (DART), a DVD-Ig, or bispecific antibody. In some aspects, the anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-1 antibody cross-competes with nivolumab for binding to human PD-1. In some aspects, the anti-PD-1 antibody binds to the same epitope as nivolumab.

In some aspects, the anti-PD-1 antibody cross-competes with pembrolizumab for binding to human PD-1. In some aspects, the anti-PD-1 antibody binds to the same epitope as pembrolizumab.

In some aspects, the anti-PD-1 antibody is nivolumab, pembrolizumab, PDR001, MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224.

In some aspects, the PD-1 pathway inhibitor is an anti-PD-L1 antibody. In some aspects, the anti-PD-L1 antibody is a full-length antibody. In some aspects, the anti-PD-L1 antibody is a monoclonal, chimeric, humanized, human, or multispecific antibody. In some aspects, the multispecific antibody is a dual-affinity re-targeting antibody (DART), a DVD-Ig, or bispecific antibody. In some aspects, the anti-PD-L₁ antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-PD-L1 antibody cross-competes with atezolizumab for binding to human PD-L1. In some aspects, the anti-PD-L1 antibody binds to the same epitope as atezolizumab.

In some aspects, the anti-PD-L1 antibody cross-competes with durvalumab for binding to human PD-L1. In some aspects, the anti-PD-L1 antibody binds to the same epitope as durvalumab.

In some aspects, the anti-PD-L1 antibody cross-competes with avelumab for binding to human PD-L1. In some aspects, the anti-PD-L1 antibody binds to the same epitope as avelumab.

In some aspects, the anti-PD-L1 antibody is BMS-936559, atezolizumab, durvalumab, avelumab, STI-1014, CX-072, KN035, LY3300054, BGB-A333, ICO 36, CK-301, or comprises an antigen binding portion thereof.

In some aspects, the PD-1 pathway inhibitor is BMS-986189.

In some aspects, the checkpoint inhibitor comprises a CTLA-4 inhibitor.

In some aspects, the CTLA-4 inhibitor is an anti-CTLA-4 antibody. In some aspects, the anti-CTLA-4 antibody is a full-length antibody. In some aspects, the anti-CTLA-4 antibody is a monoclonal, chimeric, humanized, human, or multispecific antibody. In some aspects, the multispecific antibody is a dual-affinity re-targeting antibody (DART), a DVD-Ig, or bispecific antibody. In some aspects, the anti-CTLA-4 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-CTLA-4 antibody cross-competes with ipilimumab for binding to human CTLA-4. In some aspects, the anti-CTLA-4 antibody binds to the same epitope as ipilimumab.

In some aspects, the checkpoint inhibitor is formulated for intravenous administration.

In some aspects, the LAG-3 antagonist and the checkpoint inhibitor are formulated separately. In some aspects, each checkpoint inhibitor is formulated separately when the checkpoint inhibitor comprises more than one checkpoint inhibitor.

In some aspects, the LAG-3 antagonist and the checkpoint inhibitor are formulated together. In some aspects, two or more checkpoint inhibitors are formulated together when the checkpoint inhibitor comprises more than one checkpoint inhibitor.

In some aspects, the checkpoint inhibitor is administered before the LAG-3 antagonist.

In some aspects, the LAG-3 antagonist is administered before the checkpoint inhibitor.

In some aspects, the LAG-3 antagonist and the checkpoint inhibitor are administered concurrently.

In some aspects, the checkpoint inhibitor is administered at a flat dose.

In some aspects, the checkpoint inhibitor is administered as a weight-based dose.

In some aspects, any of the above doses are administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the cancer is selected from the group consisting of breast cancer, hepatocellular cancer, gastroesophageal cancer, melanoma, bladder cancer, gastric cancer, lung cancer, kidney cancer, head and neck cancer, colon cancer, and any combination thereof. In some aspects, the cancer is a bladder cancer. In some aspects, the cancer is a gastric cancer. In some aspects, the cancer is a melanoma. In some aspects, the cancer is a lung cancer. In some aspects, the cancer is a breast cancer. In some aspects, the cancer is a hepatocellular cancer.

In some aspects, the cancer is unresectable. In some aspects, the cancer is locally advanced. In some aspects, the cancer is metastatic.

In some aspects, the administering treats the cancer. In some aspects, the administering reduces the size of a tumor associated with the cancer. In some aspects, the size of the tumor is reduced by at least about 10%, about 20%, about 30%, about 40%, or about 50% compared to the tumor size prior to the administration. In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration. In some aspects, the subject exhibits stable disease after the administration. In some aspects, the subject exhibits a partial response after the administration. In some aspects, the subject exhibits a complete response after the administration.

The present disclosure is directed to a kit for treating a subject afflicted with a tumor, the kit comprising: (a) a dosage of the LAG-3 antagonist; and (b) instructions for using the LAG-3 antagonist in the methods or uses of the present disclosure. In some aspects, the kit further comprises a dosage of a PD-1 pathway inhibitor.

### BRIEF DESCRIPTION OF THE DRAWINGS

FIG. 1 is a schematic representation of the role of LAG-3 and MHC II in T-cell exhaustion.

FIG. 2 is a schematic representation of an exemplary study design showing immunohistochemistry (IHC) staining of sectioned tumor samples for LAG-3, Pan cytokeratin (CK) (tumor), and MHC II markers, followed by digital spatial analysis of the IHC-stained slide sections by (A) scanning, (B) digital alignment and spatial analysis, and (C) registering and quantifying density, count, and proximity data across markers.

FIG. 3 is a schematic representation of an exemplary study design showing a HALO® spatial analysis workflow of IHC-stained slide sections as described in FIG. 2. Presence or absence of markers are indicated by “+” and “−” symbols, respectively. Distances between LAG-3+ T cells, also referred to as tumor infiltrating lymphocytes (“TILs”), and tumor cells is shown in micrometers (μm).

FIG. 4 is a schematic representation of an exemplary study design showing a spatial analysis workflow to determine the density of LAG-3+ TILs and the proportion of LAG-3+ TILs to MHC II+ or MHC II− tumor cells. Distances between LAG-3+ TILs and tumor cells are indicated as >30 μm or ≤30 μm. “LAG-3-D” is the density (D) of LAG-3+ TILs within ≤30 μm of MEW II+ or MEW II− tumor cells. “LAG-3-P” is the proportion (P) of LAG-3+ TILs within ≤30 μm of MHC II+vs MHC II− tumor cells.

FIG. 5 shows MEW II expression (A) in IHC-stained bladder tumor sample sections as compared to LAG-3 and (B) as a graphical representation of percentage expression on bladder and gastric tumor cells.

FIG. 6 is a graphical representation showing LAG-3-D (cells/mm²) as described for FIG. 4 in bladder and gastric tumor samples.

FIG. 7 is a graphical representation showing LAG-3-P as described for FIG. 4 as the percentage of LAG-3+ engaged (%) in bladder and gastric tumor samples.

### DETAILED DESCRIPTION OF THE DISCLOSURE

The present disclosure provides a method of treating a cancer in a human subject in need thereof comprising administering a lymphocyte activation gene-3 (LAG-3) antagonist to the subject, wherein the subject is identified as having (i) a high LAG-3 density (LAG-3-D) score, (ii) a high LAG-3 proportion (LAG-3-P) score, or (iii) both a high LAG-3-D score and a high LAG-3-P score in a tumor sample obtained from the subject. The present disclosure also provides a method of treating a cancer in a human subject in need thereof comprising (a) identifying a subject having (i) a high LAG-3-D score, (ii) a high LAG-3-P score, or (iii) both a high LAG-3-D score and a high LAG-3-P score in a tumor sample obtained from the subject and (b) administering a LAG-3 antagonist to the subject. The present disclosure also provides a method of identifying a human subject afflicted with a cancer suitable for a LAG-3 antagonist therapy, comprising computing (i) a LAG-3-D score, (ii) a LAG-3-P score, or (iii) both a LAG-3-D score and a LAG-3-P score in a tumor sample obtained from a subject in need of a LAG-3 antagonist therapy. The present disclosure also provides a LAG-3 antagonist for treating a cancer in a human subject in need thereof, wherein the subject is identified as having (i) a high LAG-3-D score, (ii) a high LAG-3-P score, or (iii) both a high LAG-3-D score and a high LAG-3-P score of a tumor sample obtained from the subject. The present disclosure also provides a LAG-3 antagonist for identifying a subject afflicted with a cancer suitable for a LAG-3 antagonist therapy, wherein (i) a LAG-3-D score, (ii) a LAG-3-P score, or (iii) both a LAG-3-D score and a LAG-3-P score in a tumor sample obtained from the subject is computed.

### I. TERMS

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

The headings provided herein are not limitations of the various aspects of the disclosure, which can be had by reference to the specification as a whole. Accordingly, the terms defined immediately below are more fully defined by reference to the specification in its entirety.

An “antagonist” shall include, without limitation, any molecule capable of blocking, reducing, or otherwise limiting an interaction or activity of a target molecule (e.g., LAG-3). In some aspects, the antagonist is an antibody. In other aspects, the antagonist comprises a small molecule. The terms “antagonist” and “inhibitor” are used interchangeably herein.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain can have the C-terminal lysine or not.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; single chain antibodies; monospecific antibodies; bispecific antibodies; and multi-specific antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, that retains the ability to bind specifically to the antigen bound by the whole immunoglobulin. Examples of an “antigen-binding portion” or “antigen-binding fragment” include: (1) a Fab fragment (fragment from papain cleavage) or a similar monovalent fragment consisting of the V_{L}, V_{H}, LC and CH1 domains; (2) a F(ab′)₂ fragment (fragment from pepsin cleavage) or a similar bivalent fragment comprising two Fab fragments linked by a disulfide bridge at the hinge region; (3) a Fd fragment consisting of the V_{H} and CH1 domains; (4) a Fv fragment consisting of the V_{L} and V_{H} domains of a single arm; (5) a single domain antibody (dAb) fragment (Ward et al., (1989) Nature 341:544-546), which consists of a V_{H} domain; (6) a bi-single domain antibody which consists of two V_{H} domains linked by a hinge (dual-affinity re-targeting antibodies (DARTs)); or (7) a dual variable domain immunoglobulin. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). These antigen-binding portions or fragments are obtained using conventional techniques known to those with skill in the art, and the portions or fragments are screened for utility in the same manner as are intact antibodies. Antigen-binding portions or fragments can be produced by recombinant DNA techniques, or by enzymatic or chemical cleavage of intact immunoglobulins.

An “isolated antibody” refers to an antibody that is substantially free of other antibodies having different antigenic specificities (e.g., an isolated antibody that binds specifically to LAG-3 is substantially free of antibodies that bind specifically to antigens other than LAG-3). An isolated antibody that binds specifically to LAG-3 may, however, have cross-reactivity to other antigens, such as LAG-3 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-LAG-3 antibody binds specifically to LAG-3, an anti-PD-1 antibody binds specifically to PD-1, an anti-PD-L1 antibody binds specifically to PD-L1, and an anti-CTLA-4 antibody binds specifically to CTLA-4.

“LAG-3” refers to Lymphocyte Activation Gene-3. The term “LAG-3” includes variants, isoforms, homologs, orthologs and paralogs. For example, antibodies specific for a human LAG-3 protein can, in certain cases, cross-react with a LAG-3 protein from a species other than human. In other aspects, the antibodies specific for a human LAG-3 protein can be completely specific for the human LAG-3 protein and not exhibit species or other types of cross-reactivity, or can cross-react with LAG-3 from certain other species, but not all other species (e.g., cross-react with monkey LAG-3 but not mouse LAG-3). The term “human LAG-3” refers to human sequence LAG-3, such as the complete amino acid sequence of human LAG-3 having GenBank Accession No. NP_002277. The term “mouse LAG-3” refers to mouse sequence LAG-3, such as the complete amino acid sequence of mouse LAG-3 having GenBank Accession No. NP_032505. LAG-3 is also known in the art as, for example, CD223. The human LAG-3 sequence can differ from human LAG-3 of GenBank Accession No. NP_002277 by having, e.g., conserved mutations or mutations in non-conserved regions, and the LAG-3 has substantially the same biological function as the human LAG-3 of GenBank Accession No. NP_002277. For example, a biological function of human LAG-3 is having an epitope in the extracellular domain of LAG-3 that is specifically bound by an antibody of the instant disclosure or a biological function of human LAG-3 is binding to MHC Class II molecules.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

The terms “Programmed Death Ligand-2” and “PD-L2” as used herein include human PD-L2 (hPD-L2), variants, isoforms, and species homologs of hPD-L2, and analogs having at least one common epitope with hPD-L2. The complete hPD-L2 sequence can be found under GenBank Accession No. Q9BQ51.

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

“T cell Immunoglobulin and Mucin domain-3” (TIM-3), also known as hepatitis A virus cellular receptor 2 (HAVCR2), refers to a type-I transmembrane protein that was initially identified on activated IFN-γ producing T cells (e.g., type 1 helper CD4+ T cells and cytotoxic CD8+ T cells) and shown to induce T cell death or exhaustion after binding to galectin-9. The term “TIM-3” as used herein includes human TIM-3 (hTIM-3), variants, isoforms, and species homologs of hTIM-3, and analogs having at least one common epitope with hTIM-3. Two isoforms of hTIM-3 have been identified. Isoform 1 (GenBank Accession No. NP_116171) consists of 301 amino acids and represents the canonical sequence. Isoform 2 (GenBank Accession No. AAH20843) consists of 142 amino acids, and is soluble.

The term “LAG-3 positive” or “LAG-3 expression positive,” relating to LAG-3 expression, refers to the proportion of cells in a test tissue sample comprising tumor cells and tumor-infiltrating inflammatory cells above which the tissue sample is scored as expressing LAG-3.

“LAG-3 negative” or “LAG-3 expression negative,” refers to the proportion of cells in a test tissue sample comprising tumor cells and tumor-infiltrating inflammatory cells that are not LAG-3 positive or LAG-3 expression positive.

The term “PD-L1 positive” or “PD-L1 expression positive,” relating to cell surface PD-L1 expression, refers to the proportion of cells in a test tissue sample comprising tumor cells and tumor-infiltrating inflammatory cells above which the sample is scored as expressing cell surface PD-L1.

The term “PD-L1 negative” or “PD-L1 expression negative,” relating to cell surface PD-L1 expression, refers to the proportion of cells in a test tissue sample comprising tumor cells and tumor-infiltrating inflammatory cells that are not PD-L1 positive or PD-L1 expression positive.

The term “tumor mutation burden” (TMB) as used herein refers to the number of somatic mutations in a tumor's genome and/or the number of somatic mutations per area of the tumor's genome. Germline (inherited) variants are excluded when determining TMB, because the immune system has a higher likelihood of recognizing these as self. Tumor mutation burden (TMB) can also be used interchangeably with “tumor mutation load,” “tumor mutational burden,” or “tumor mutational load.”

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

“Administering” refers to the physical introduction of a composition comprising a therapeutic agent to a subject, using any of the various methods and delivery systems known to those skilled in the art. Preferred routes of administration for the immunotherapy, e.g., a LAG-3 antagonist, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, in some aspects, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Response Evaluation Criteria In Solid Tumors (RECIST) is a measure for treatment efficacy and are established rules that define when tumors respond, stabilize, or progress during treatment. RECIST 1.1 is the current guideline to solid tumor measurement and definitions for objective assessment of change in tumor size for use in adult and pediatric cancer clinical trials.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment can refer to alleviation of at least one symptom of a solid tumor. Such effective treatment can, e.g., reduce patient pain, reduce the size and/or number of lesions, can reduce or prevent metastasis of a tumor, and/or can slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-LAG-3 antibody alone or the amount of anti-LAG-3 antibody and the amount an additional therapeutic agent (e.g., anti-PD-1 antibody), in combination, clinically proven to affect a significant decrease in cancer or slowing of progression of cancer, such as an advanced solid tumor.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., an amount in μg or mg).

The use of the term “fixed dose combination” with regard to a composition of the invention means that two or more different inhibitors as described herein (e.g., an anti-LAG-3 antibody and an anti-PD-1 antibody) in a single composition are present in the composition in particular (fixed) ratios with each other. In some aspects, the fixed dose is based on the weight (e.g., mg) of the inhibitors. In certain aspects, the fixed dose is based on the concentration (e.g., mg/ml) of the inhibitors. In some aspects, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first inhibitor to mg second inhibitor. For example, the 3:1 ratio of a first antibody and a second antibody can mean that a vial can contain about 240 mg of the first antibody and 80 mg of the second antibody or about 3 mg/ml of the first antibody and 1 mg/ml of the second antibody.

The term “weight based dose” as referred to herein means that a dose that is administered to a patient is calculated based on the weight of the patient. For example, when a patient with 60 kg body weight requires 3 mg/kg of an anti-LAG-3 antibody in combination with 3 mg/kg of an anti-PD-1 antibody, one can draw the appropriate amounts of the anti-LAG-3 antibody (i.e., 180 mg) and the anti-PD-1 antibody (i.e., 180 mg) at once from a 1:1 ratio fixed dose combination of an anti-LAG3 antibody and an anti-PD-1 antibody.

“Dosing interval,” as used herein, means the amount of time that elapses between multiple doses of a formulation disclosed herein being administered to a subject. Dosing interval can thus be indicated as ranges.

The term “dosing frequency” as used herein refers to the frequency of administering doses of a formulation disclosed herein in a given time. Dosing frequency can be indicated as the number of doses per a given time, e.g., once a week or once in two weeks, etc.

The terms “about once a week,” “once about every week,” “once about every two weeks,” or any other similar dosing interval terms as used herein means approximate number, and “about once a week” or “once about every week” can include every seven days±two days, i.e., every five days to every nine days. The dosing frequency of “once a week” thus can be every five days, every six days, every seven days, every eight days, or every nine days. “Once about every three weeks” can include every 21 days±3 days, i.e., every 25 days to every 31 days. Similar approximations apply, for example, to once about every two weeks, once about every four weeks, once about every five weeks, once about every six weeks, once about every seven weeks, once about every eight weeks, once about every nine weeks, once about every ten weeks, once about every eleven weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

The term “tumor” as used herein refers to any mass of tissue that results from excessive cell growth or proliferation, either benign (non-cancerous) or malignant (cancerous), including pre-cancerous lesions.

By way of example, an “anti-cancer agent” promotes cancer regression in a subject. In preferred aspects, a therapeutically effective amount of the drug promotes cancer regression to the point of eliminating the cancer. “Promoting cancer regression” means that administering an effective amount of the anti-cancer agent, alone or in combination with another agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the agent.

By way of example for the treatment of tumors, a therapeutically effective amount of an anti-cancer agent preferably inhibits cell growth or tumor growth by at least about 20%, at least about 40%, at least about 60%, or at least about 80% relative to untreated subjects. In other aspects of the disclosure, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for immune-related response patterns.

As used herein, an “immuno-oncology” therapy or an “I-O” or “IO” therapy refers to a therapy that comprises utilizing an immune response to target and treat a tumor in a subject. As such, as used herein, an I-O therapy is a type of anti-cancer therapy. In some aspects, and I-O therapy comprises administering an antibody or an antigen-binding fragment thereof to a subject. In some aspects, an I-O therapy comprises administering to a subject an immune cell, e.g., a T cell, e.g., a modified T cell, e.g., a T cell modified to express a chimeric antigen receptor or a particular T cell receptor. In some aspects, the I-O therapy comprises administering a therapeutic vaccine to a subject. In some aspects, the I-O therapy comprises administering a cytokine or a chemokine to a subject. In some aspects, the I-O therapy comprises administering an interleukin to a subject. In some aspects, the I-O therapy comprises administering an interferon to a subject. In some aspects, the I-O therapy comprises administering a colony stimulating factor to a subject.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “tumor-infiltrating inflammatory cell” or “tumor-associated inflammatory cell” is any type of cell that typically participates in an inflammatory response in a subject and which infiltrates tumor tissue. Such cells include tumor-infiltrating lymphocytes (TILs), macrophages, monocytes, eosinophils, histiocytes and dendritic cells.

The term “tumor sample” as used herein refers to tumor material isolated from a tumor of a subject. The tumor sample can contain any portion of a tumor suitable for determining target protein expression (e.g., LAG-3, MEW class II, and/or tumor antigen), for example, by immunohistochemistry (IHC). In one aspect, the tumor sample is a tumor tissue biopsy, e.g., a formalin-fixed, paraffin-embedded (FFPE) tumor tissue or a fresh-frozen tumor tissue or the like. In another aspect, the tumor sample can be sectioned into multiple tumor sections. In other aspects, the tumor sample is serially sectioned into multiple sections.

Various aspects of the disclosure are described in further detail in the following subsections.

### II. METHODS OF THE DISCLOSURE

Provided herein are methods of increasing efficacy of a LAG-3 antagonist therapy by identifying or selecting a human subject or a human subject population suitable or responsive for the LAG-3 antagonist therapy. The methods provided herein are directed to quantitative spatial profiling of human subjects who are suitable or responsive for a LAG-3 antagonist therapy. In one aspect, the present disclosure is directed to a method of treating a cancer, e.g., reducing volume and/or growth of a tumor, in a human subject in need thereof comprising administering a lymphocyte activation gene-3 (LAG-3) antagonist to the subject, wherein the subject is identified as having (i) a high LAG-3 density (LAG-3-D) score, (ii) a high LAG-3 proportion (LAG-3-P) score, or (iii) both a high LAG-3-D score and a high LAG-3-P score in a tumor sample obtained from the subject.

In another aspect, the present disclosure is directed to a method of treating a cancer in a human subject in need thereof comprising (a) identifying a subject having (i) a high LAG-3-D score, (ii) a high LAG-3-P score, or (iii) both a high LAG-3-D score and a high LAG-3-P score in a tumor sample obtained from the subject and (b) administering a LAG-3 antagonist to the subject.

In another aspect, the present disclosure is directed to a method of identifying or selecting a human subject afflicted with a cancer suitable for a LAG-3 antagonist therapy, comprising computing (i) a LAG-3-D score, (ii) a LAG-3-P score, or (iii) both a LAG-3-D score and a LAG-3-P score in a tumor sample obtained from a subject in need of a LAG-3 antagonist therapy. In some aspects, the subject exhibits a high LAG-3-D score, a high LAG-3-P score, or both a high LAG-3-D score and a high LAG-3-P score. In some aspects, the method further comprises administering a LAG-3 antagonist to the subject. In some aspects, the subject identified or selected by the present method is responsive to a LAG-3 antagonist therapy.

In another aspect, the present disclosure is directed to a LAG-3 antagonist for treating a cancer in a human subject in need thereof, wherein the subject is identified as having (i) a high LAG-3-D score, (ii) a high LAG-3-P score, or (iii) both a high LAG-3-D score and a high LAG-3-P score of a tumor sample obtained from the subject.

In another aspect, the present disclosure is directed to a LAG-3 antagonist for identifying or selecting a subject afflicted with a cancer suitable for a LAG-3 antagonist therapy, wherein (i) a LAG-3-D score, (ii) a LAG-3-P score, or (iii) both a LAG-3-D score and a LAG-3-P score in a tumor sample obtained from the subject is computed. In some aspects, the subject exhibits a high LAG-3-D score, a high LAG-3-P score, or both a high LAG-3-D score and a high LAG-3-P score. In some aspects, the subject identified or selected by the present method is responsive to a LAG-3 antagonist therapy.

### II.A. LAG-3-D Scores and LAG-3-P Scores

The LAG-3-D score according to the disclosure can be determined by measuring a density of T cells expressing LAG-3 in proximity to one or more tumor cells expressing major histocompatibility complex class II (MHC II) in the tumor sample. In some aspects, the LAG-3-D score is calculated as (i) the number of the T cells expressing LAG-3 in proximity to the tumor cells expressing MHC II divided by (ii) the tumor area (mm²) of the tumor sample.

The LAG-3-P score according to the disclosure can be determined by measuring a proportion of T cells expressing LAG-3 in proximity to one or more tumor cells expressing MHC II in the tumor sample. In some aspects, the LAG-3-P score is calculated as (i) the number of the T cells expressing LAG-3 in proximity to the tumor cell expressing MHC II divided by (ii) the total number of T cells expressing LAG-3 in the tumor sample.

In some aspects, the proximity is between the cell surface of a T cell expressing LAG-3 and the cell surface of a tumor cell expressing MHC II.

In some aspects, the proximity is between the nucleus of a T cell expressing LAG-3 and the nucleus of a tumor cell expressing MHC II.

In some aspects, the proximity is between the LAG-3 and the MHC class II and/or between the LAG-3 and a tumor antigen expressed on the tumor cells.

In some aspects, the proximity is equal to or less than about 50 μm, equal to or less than about 45 μm, equal to or less than about 40 μm, equal to or less than about 35 μm, or equal to or less than about 30 μm. In some aspects, the proximity is equal to or less than about 50 μm. In some aspects, the proximity is equal to or less than about 40 μm. In some aspects, the proximity is equal to or less than about 35 μm. In some aspects, the proximity is equal to or less than about 30 μm.

In some aspects, the tumor sample comprises one or more tumor sections derived from a tumor tissue biopsy or a tumor tissue resection. In some aspects, the one or more tumor sections comprise a formalin-fixed, paraffin-embedded tumor tissue or a fresh-frozen tumor tissue. In some aspects, the one or more tumor sections comprise serially sectioned tumor sections. In some aspects, the one or more tumor sections are stained by immunohistochemistry (IHC). In some aspects, the one or more tumor sections comprise one tumor section, two tumor sections, three tumor sections, four tumor sections, five tumor sections, six tumor sections, seven tumor sections, eight tumor sections, nine tumor sections, ten tumor sections, 11 tumor sections, 12 tumor sections, 13 tumor sections, 14 tumor sections, 15 tumor sections, 16 tumor sections, 17 tumor sections, 18 tumor sections, 19 tumor sections, 20 tumor sections, 21 tumor sections, 22 tumor sections, 23 tumor sections, 24 tumor sections, 25 tumor sections, 26 tumor sections, 27 tumor sections, 28 tumor sections, 29 tumor sections, or 30 tumor sections. In some aspects, the one or more tumor sections comprise three tumor sections. In some aspects, the one or more tumor sections comprise 15 tumor sections. In some aspects, the one or more tumor sections comprise 18 tumor sections. In some aspects, the one or more tumor sections comprise 20 tumor sections. In some aspects, the one or more tumor sections comprise 21 tumor sections.

In some aspects, one tumor section of the tumor sample is stained for the LAG-3 and/or the MHC II. In some aspects, the tumor section is further stained for a tumor antigen. In some aspects, the tumor antigen is Pan cytokeratin (CK). In some aspects, one tumor section for the tumor sample is stained for all three markers, i.e., LAG-3, MHC class II, and tumor antigen (e.g., Pan CK).

In some aspects, the tumor sample comprises a first tumor section stained for the LAG-3, a second tumor section stained for the MHC II, and a third tumor section stained for a tumor antigen. In some aspects, the first tumor section, the second tumor section, and the third tumor section are serially sectioned from the tumor sample. In some aspects, the tumor sample comprises a first group of tumor sections (e.g., 2, 3, 4, 5, 6, 7, 8, 9, or 10 tumor sections) stained for the LAG-3, a second group of tumor sections (e.g., 2, 3, 4, 5, 6, 7, 8, 9, or 10 tumor sections) stained for the MHC II, and a third group of tumor sections (e.g., 2, 3, 4, 5, 6, 7, 8, 9, or 10 tumor sections) stained for a tumor antigen. In some aspects, the first group, the second group, and the third group of tumor sections are serially sectioned from the tumor sample.

In some aspects, LAG-3-D scores and LAG-3-P scores are determined by quantitative spatial profiling. In some aspects, the quantitative spatial profiling is digital spatial analysis.

In some aspects, the high LAG-3-D score is at least about 5 cells/mm², at least about 6 cells/mm², at least about 7 cells/mm², at least about 8 cells/mm², at least about 9 cells/mm², at least about 10 cells/mm², at least about 11 cells/mm², at least about 12 cells/mm², at least about 13 cells/mm², at least about 14 cells/mm², at least about 15 cells/mm², at least about 16 cells/mm², at least about 17 cells/mm², at least about 18 cells/mm², at least about 19 cells/mm², at least about 20 cells/mm², at least about 25 cells/mm², at least about 30 cells/mm², at least about 35 cells/mm², at least about 40 cells/mm², at least about 45 cells/mm², at least about 50 cells/mm², at least about 55 cells/mm², at least about 60 cells/mm², at least about 65 cells/mm², at least about 70 cells/mm², at least about 75 cells/mm², at least about 80 cells/mm², at least about 85 cells/mm², at least about 90 cells/mm², at least about 95 cells/mm², or at least about 100 cells/mm². In some aspects, the high LAG-3-D score is at least about 5 cells/mm². In some aspects, the high LAG-3-D score is at least about 10 cells/mm². In some aspects, the high LAG-3-D score is at least about 15 cells/mm². In some aspects, the high LAG-3-D score is at least about 20 cells/mm², In some aspects, the high LAG-3-D score is at least about 25 cells/mm². In some aspects, the high LAG-3-D score is at least about 30 cells/mm², In some aspects, the high LAG-3-D score is at least about 35 cells/mm². In some aspects, the high LAG-3-D score is at least about 40 cells/mm². In some aspects, the high LAG-3-D score is at least about 45 cells/mm². In some aspects, the high LAG-3-D score is at least about 50 cells/mm², In some aspects, the high LAG-3-D score is at least about 55 cells/mm². In some aspects, the high LAG-3-D score is at least about 60 cells/mm². In some aspects, the high LAG-3-D score is at least about 65 cells/mm². In some aspects, the high LAG-3-D score is at least about 70 cells/mm². In some aspects, the high LAG-3-D score is at least about 75 cells/mm². In some aspects, the high LAG-3-D score is at least about 80 cells/mm². In some aspects, the high LAG-3-D score is at least about 85 cells/mm². In some aspects, the high LAG-3-D score is at least about 90 cells/mm². In some aspects, the high LAG-3-D score is at least about 95 cells/mm². In some aspects, the high LAG-3-D score is at least about 100 cells/mm². 101431 In some aspects, the high LAG-3-P score is at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some aspects, the high LAG-3-P score is between at least about 40% and about 100%. In some aspects, the high LAG-3-P score is at least about 40%. In some aspects, the high LAG-3-P score is at least about 45%. In some aspects, the high LAG-3-P score is at least about 50%. In some aspects, the high LAG-3-P score is at least about 55%. In some aspects, the high LAG-3-P score is at least about 60%. In some aspects, the high LAG-3-P score is at least about 65%. In some aspects, the high LAG-3-P score is at least about 70%. In some aspects, the high LAG-3-P score is at least about 75%. In some aspects, the high LAG-3-P score is at least about 80%. In some aspects, the high LAG-3-P score is at least about 85%. In some aspects, the high LAG-3-P score is at least about 90%. In some aspects, the high LAG-3-P score is at least about 95%. In some aspects, the high LAG-3-P score is at least about 100%. In some aspects, the high LAG-3-P score is between at least about 40% and about 100%. In some aspects, the high LAG-3-P score is between at least about 50% and about 100%. In some aspects, the high LAG-3-P score is between at least about 60% and about 100%. In some aspects, the high LAG-3-P score is between at least about 70% and about 100%. In some aspects, the high LAG-3-P score is between at least about 80% and about 100%. In some aspects, the high LAG-3-P score is between at least about 90% and about 100%.

### II.B. LAG-3 Antagonists and Combination Therapies

In some aspects, the present disclosure is directed to methods and uses for treating a cancer in a human subject in need thereof comprising administering a LAG-3 antagonist to the subject. In some aspects, the subject is administered a LAG-3 antagonist monotherapy, e.g., wherein the subject is not administered one or more additional therapeutic agent (e.g., an anti-cancer agent).

In some aspects, the subject is administered a combination therapy, e.g., wherein the subject is administered a LAG-3 antagonist and one or more additional therapeutic agent (e.g., an anti-cancer agent).

In some aspects, the methods and uses for treating a cancer further comprise administering an additional checkpoint inhibitor to the subject. In some aspects, the checkpoint inhibitor comprises an anti-PD-1 antibody, an anti-CTLA-4 antibody, an anti-TIM3 antibody, an anti-PD-L1 antibody, or any combination thereof. In some aspects, the checkpoint inhibitor comprises an anti-PD-1 antibody. In some aspects, the checkpoint inhibitor comprises an anti-PD-L1 antibody.

### II.B.1. LAG-3 Antagonists

As used herein a LAG-3 antagonist includes, but is not limited to, LAG-3 binding agents, e.g., a LAG-3 antibody, and soluble LAG-3 polypeptides, e.g., a fusion protein comprising the extracellular portion of LAG-3. The term “LAG-3 antagonist” as used herein is interchangeable with the term “LAG-3 inhibitor.”

In some aspects, the LAG-3 antagonist is a soluble LAG-3 polypeptide. In some aspects, the soluble LAG-3 polypeptide is a fusion polypeptide, e.g., a fusion protein comprising the extracellular portion of LAG-3. In some aspects, the soluble LAG-3 polypeptide is a LAG-3-Fc fusion polypeptide capable of binding to WIC Class II. In some aspects, the soluble LAG-3 polypeptide comprises a ligand binding fragment of the LAG-3 extracellular domain. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha). See, e.g., Brignone C, et al., J. Immunol. (2007); 179:4202-4211 and WO2009/044273.

In some aspects, the LAG-3 antagonist is an anti-LAG-3 antibody.

Anti-LAG-3 antibodies (or VH/VL domains derived therefrom) suitable for use herein can be generated using methods well known in the art. Alternatively, art recognized anti-LAG-3 antibodies can be used. Antibodies that bind to LAG-3 have been disclosed, for example, in Int'l Publ. No. WO/2015/042246 and U.S. Publ. Nos. 2014/0093511 and 2011/0150892, each of which is incorporated by reference herein in its entirety.

An exemplary LAG-3 antibody useful in the present disclosure is 25F7 (described in U.S. Publ. No. 2011/0150892). An additional exemplary LAG-3 antibody useful in the present disclosure is BMS-986016 (relatlimab). In some aspects, an anti-LAG-3 antibody useful in the present disclosure cross-competes with 25F7 or BMS-986016 (relatlimab) for binding to human LAG-3. In some aspects, an anti-LAG-3 antibody useful in the present disclosure binds to the same epitope as 25F7 or BMS-986016 (relatlimab).

Other art-recognized anti-LAG-3 antibodies that can be used in the methods and for the uses of the disclosure include IMP731 (H5L7BW) described in US 2011/007023, MK-4280 (28G-10) described in WO2016028672, REGN3767 (fianlimab) described in Burova E, et al., J. Immunother. Cancer (2016); 4 (Supp. 1): P195, humanized BAP050 described in WO2017/019894, GSK2831781, IMP-701 (LAG-525; ieramilimab), aLAG3(0414), aLAG3(0416), Sym022, TSR-033, TSR-075, XmAb22841, MGD013, BI754111, FS118, P 13B02-30, AVA-017 and AGEN1746. These and other anti-LAG-3 antibodies useful in the claimed invention can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

Anti-LAG-3 antibodies that can be used in the methods and for the uses of the disclosure also include isolated antibodies that bind specifically to human LAG-3 and cross-compete for binding to human LAG-3 with any anti-LAG-3 antibody disclosed herein, e.g., relatlimab. In some aspects, the anti-LAG-3 antibody binds the same epitope as any of the anti-LAG-3 antibodies described herein, e.g., relatlimab.

In some aspects, the antibodies that cross-compete for binding to human LAG-3 with, or bind to the same epitope region as, any anti-LAG-3 antibody disclosed herein, e.g., relatlimab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

The ability of antibodies to cross-compete for binding to an antigen indicates that the antibodies bind to the same epitope region of the antigen and sterically hinder the binding of other cross-competing antibodies to that particular epitope region. These cross-competing antibodies are expected to have functional properties very similar those of the reference antibody, e.g., relatlimab, by virtue of their binding to the same epitope region. Cross-competing antibodies can be readily identified based on their ability to cross-compete in standard binding assays such as Biacore analysis, ELISA assays or flow cytometry (see, e.g., WO 2013/173223).

Anti-LAG-3 antibodies that can be used in the methods and for the uses of the disclosure also include antigen-binding portions of any of the above full-length antibodies. It has been amply demonstrated that the antigen-binding function of an antibody can be performed by fragments of a full-length antibody.

Biosimilars of any of the anti-LAG-3 antibodies disclosed herein can also be used in the methods and for the uses of the disclosure.

In other aspects, the anti-LAG-3 antibody has the heavy and light chain CDRs or variable regions of any of the anti-LAG-3 antibodies disclosed herein, e.g., relatlimab. Accordingly, in one aspect, the antibody comprises CDR1, CDR2, and CDR3 domains of the VH region of an anti-LAG-3 antibody disclosed herein, e.g., relatlimab, and CDR1, CDR2 and CDR3 domains of the VL region of the antibody, e.g., relatlimab. In another aspect, the anti-LAG-3 antibody comprises VH and/or VL regions of any of the anti-LAG-3 antibodies disclosed herein, e.g., relatlimab.

In some aspects, the anti-LAG-3 antibody is a full-length antibody.

In some aspects, the anti-LAG-3 antibody is a monoclonal, chimeric, humanized, human, or multispecific antibody. In some aspects, the multispecific antibody is a dual-affinity re-targeting antibody (DART), a DVD-Ig, or bispecific antibody.

In some aspects, the anti-LAG-3 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody is BMS-986016 (relatlimab), LAG-525 (IMP-701, ieramilimab), MK-4280 (28G-10), REGN3767 (fianlimab), TSR-033, TSR-075, Sym022, FS-118, IMP731 (H5L7BW), GSK2831781, humanized BAP050, aLAG3(0414), aLAG3(0416), XmAb22841, MGD013, BI754111, P 13B02-30, AVA-017, 25F7, AGEN1746, or comprises an antigen binding portion thereof.

In certain aspects, an anti-LAG-3 antibody is used to determine LAG-3 expression. In some aspects, an anti-LAG-3 antibody is selected for its ability to bind to LAG-3 in formalin-fixed, paraffin-embedded (FFPE) tissue specimens. In other aspects, an anti-LAG-3 antibody is capable of binding to LAG-3 in frozen tissues. In further aspects, an anti-LAG-3 antibody is capable of distinguishing membrane bound, cytoplasmic, and/or soluble forms of LAG-3.

In some aspects, an anti-LAG-3 antibody useful for assaying, detecting, and/or quantifying LAG-3 expression in accordance with the methods described herein is the 17B4 mouse IgG1 anti-human LAG-3 monoclonal antibody, or an antigen binding fragment thereof. See, e.g., Matsuzaki, J et al.; PNAS 107, 7875 (2010).

### II.B.2 Additional Therapeutic Agents and Therapies

In some aspects, the methods and uses of the disclosure further comprise administering to the subject an additional therapeutic agent and/or anti-cancer therapy.

The additional anti-cancer therapy can comprise any therapy known in the art for the treatment of a tumor in a subject and/or any standard-of-care therapy, as disclosed herein. In some aspects, the additional anti-cancer therapy comprises a surgery, a radiation therapy, a chemotherapy, an immunotherapy, or any combination thereof. In some aspects, the additional anti-cancer therapy comprises a chemotherapy, including any chemotherapeutic agent disclosed herein. In some aspects, the chemotherapy comprises platinum-doublet chemotherapy.

In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor comprises sorafenib (e.g., sorafenib tosylate, also known as NEXAVAR®), lenvatinib (e.g., lenvatinib mesylate, also known as LENVIMA®), regorafenib (e.g., STIVARGA®), cabozantinib (e.g., cabozantinib S-malate, also known as CABOMETYX®), sunitinib (e.g., sunitinib malate, also known as SUTENT®), brivanib, linifanib, erlotinib (e.g., erlotinib hydrocholoride, also known as TARCEVA®), pemigatinib (also known as PEMAZYRE™), everolimus (also known as AFINITOR® or ZORTRESS®), gefitinib (IRESSA®), imatinib (e.g., imatinib mesylate), lapatinib (e.g., lapatinib ditosylate, also known as TYKERB®), nilotinib (e.g., nioltinib hydrochloride, also known as TASIGNA®), pazopanib (e.g., pazopanib hydrochloride, also known as VOTRIENT®), temsirolimus (also known as TORISEL®), or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof. In some aspects, the anti-angiogenesis agent comprises bevacizumab (also known as AVASTIN®), ramucirumab (also known as CYRAMZA®), aflibercept (also known as EYLEA® or ZALTRAP®), tanibirumab, olaratumab (also known as LARTRUVO™), nesvacumab, AMG780, MEDI3617, vanucizumab, rilotumumab, ficlatuzumab, TAK-701, onartuzumab, emibetuzumab, or any combination thereof.

In some aspects, the checkpoint stimulator comprises an agonist of B7-1, B7-2, CD28, 4-1BB (CD137), 4-1BBL, GITR, inducible T cell co-stimulator (ICOS), ICOS-L, OX40, OX40L, CD70, CD27, CD40, death receptor 3 (DR3), CD28H, or any combination thereof.

In some aspects, the chemotherapeutic agent comprises an alkylating agent, an antimetabolite, an antineoplastic antibiotic, a mitotic inhibitor, a hormone or hormone modulator, a protein tyrosine kinase inhibitor, an epidermal growth factor inhibitor, a proteasome inhibitor, other neoplastic agent, or any combination thereof.

In some aspects, the immunotherapeutic agent comprises an antibody that specifically ICOS, CD137 (4-1BB), CD134 (OX40), NKG2A, CD27, CD96, GITR, Herpes Virus Entry Mediator (HVEM), PD-1, PD-L1, CTLA-4, BTLA, TIM-3, A2aR, Killer cell Lectin-like Receptor G1 (KLRG-1), Natural Killer Cell Receptor 2B4 (CD244), CD160, TIGIT, VISTA, KIR, TGFβ, IL-10, IL-8, B7-H4, Fas ligand, CSF1R, CXCR4, mesothelin, CEACAM-1, CD52, HER2, MICA, MICB, or any combination thereof.

In some aspects, the platinum agent comprises cisplatin, carboplatin, oxaliplatin, satraplatin, picoplatin, nedaplatin, triplatin (e.g., triplatin tetranitrate), lipoplatin, phenanthriplatin, or any combination thereof.

In some aspects, the alkylating agent comprises altretamine, bendamustine, busulfan, carboplatin, carmustine, chlorambucil, cisplatin, cyclophosphamide, dacarbazine, ifosfamide, lomustine, mechlorethamine, melphalan, oxaliplatin, procarbazine, streptozocin, temozolomide, thiotepa, or any combination thereof.

In some aspects, the taxane comprises paclitaxel, albumin-bound paclitaxel, docetaxel, cabazitaxel, or any combination thereof.

In some aspects, the nucleoside analog comprises cytarabine, gemcitabine, lamivudine, entecavir, telbivudine, or any combination thereof.

In some aspects, the antimetabolite comprises capecitabine, cladribine, clofarabine, cytarabine, floxuridine, fludarabine, fluorouracil, gemcitabine, mercaptopurine, methotrexate, pemetrexed, pentostatin, pralatrexate, thioguanine, or any combination thereof.

In some embodiments, the topoisomerase inhibitor comprises etoposide, mitoxantrone, doxorubicin, irinotecan, topotecan, camptothecin, or any combination thereof.

In some aspects, the anthracycline is doxorubicin, daunorubicin, epirubicin, idarubicin, or any combination thereof.

In some aspects, the vinca alkaloid is vinblastine, vincristine, vinorelbine, vindesine, vincaminol, vineridine, vinburnine, or any combination thereof.

### II.B.3 Checkpoint Inhibitors

In some aspects, the anti-cancer agent that is administered as an additional therapeutic agent in the methods of the disclosure is a checkpoint inhibitor.

In some aspects, the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

### II.B.4. PD-1 Pathway Inhibitors

In some aspects, the checkpoint inhibitor for use in the methods and for the uses of the disclosure comprises a PD-1 pathway inhibitor.

In some aspects, the PD-1 pathway inhibitor is a PD-1 inhibitor and/or a PD-L1 inhibitor.

In some aspects, the PD-1 inhibitor and/or PD-L1 inhibitor is a small molecule.

In some aspects, the PD-1 inhibitor and/or PD-L1 inhibitor is a millamolecule.

In some aspects, the PD-1 inhibitor and/or PD-L1 inhibitor is a macrocyclic peptide.

In certain aspects, the PD-1 inhibitor and/or PD-L1 inhibitor is BMS-986189.

In some aspects, the PD-1 inhibitor is an inhibitor disclosed in International Publication No. WO2014/151634, which is incorporated by reference herein in its entirety.

In some aspects, the PD-1 inhibitor is INCMGA00012 (Insight Pharmaceuticals).

In some aspects, the PD-1 inhibitor comprises a combination of an anti-PD-1 antibody disclosed herein and a PD-1 small molecule inhibitor.

In some aspects, the PD-L1 inhibitor comprises a millamolecule having a formula set forth in formula (I):

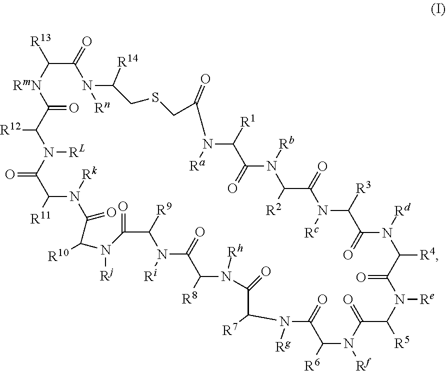

wherein R¹-R¹³ are amino acid side chains, R^{a}-Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In some aspects, the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224 (see, e.g., US 2013/0017199).

In some aspects, the PD-1 pathway inhibitor is an anti-PD-1 antibody and/or an anti-PD-L1 antibody.

### II.B.4.a Anti-PD-1 Antibodies

Anti-PD-1 antibodies that are known in the art can be used in the methods and uses of the disclosure. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics. 101971 Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; also known as spartalizumab; see WO 2015/112900), MEDI-0680 (AstraZeneca; also known as AMP-514; see WO 2012/145493), cemiplimab (Regeneron; also known as LIBTAYO® or REGN-2810; see WO 2015/112800), JS001 (TAIZHOU JUNSHI PHARMA; also known as toripalimab; see Si-Yang Liu et al., J. Hematol. Oncol. 10:136 (2017)), PF-06801591 (Pfizer; also known as sasanlimab; US 2016/0159905), BGB-A317 (Beigene; also known as tislelizumab; see WO 2015/35606 and US 2015/0079109), BI 754091 (Boehringer Ingelheim; see Zettl M et al., Cancer. Res. (2018); 78 (13 Suppl): Abstract 4558), INCSHR1210 (Jiangsu Hengrui Medicine; also known as SHR-1210 or camrelizumab; see WO 2015/085847; Si-Yang Liu et al., J. Hematol. Oncol. 10:136 (2017)), TSR-042 (Tesaro Biopharmaceutical; also known as ANB011 or dostarlimab; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), I131308 (Innovent; also known as sintilimab; see WO 2017/024465, WO 2017/025016, WO 2017/132825, and WO 2017/133540), and and SSI-361 (Lyvgen Biopharma Holdings Limited, US 2018/0346569).

Anti-PD-1 antibodies that can be used in the methods and for the uses of the disclosure also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab.

In some aspects, the antibodies that cross-compete for binding to human PD-1 with, or bind to the same epitope region as, any anti-PD-1 antibody disclosed herein, e.g., nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies that can be used in the methods of the disclosure also include antigen-binding portions of any of the above full-length antibodies.

Anti-PD-1 antibodies that can be used in the methods of the disclosure are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

Pembrolizumab is a humanized monoclonal IgG4 (S228P) antibody directed against human cell surface receptor PD-1. Pembrolizumab is described, for example, in U.S. Pat. Nos. 8,354,509 and 8,900,587.

Biosimilars of any of the anti-PD-1 antibodies disclosed herein can also be used in the methods and for the uses of the disclosure.

In other aspects, the anti-PD-1 antibody has the heavy and light chain CDRs or variable regions of any of the anti-PD-1 antibodies disclosed herein, e.g., nivolumab. Accordingly, in one aspect, the antibody comprises CDR1, CDR2, and CDR3 domains of the VH region of an anti-PD-1 antibody disclosed herein, e.g., nivolumab, and CDR1, CDR2 and CDR3 domains of the VL region of the antibody, e.g., nivolumab. In another aspect, the anti-PD-1 antibody comprises VH and/or VL regions of any of the anti-PD-1 antibodies disclosed herein, e.g., nivolumab.

In some aspects, the anti-PD-1 antibody is a full-length antibody.

In some aspects, the anti-PD-1 antibody is a monoclonal, chimeric, humanized, human, or multispecific antibody. In some aspects, the multispecific antibody is a dual-affinity re-targeting antibody (DART), a DVD-Ig, or bispecific antibody.

In some aspects, the anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-1 antibody cross-competes with nivolumab for binding to human PD-1.

In some aspects, the anti-PD-1 antibody binds to the same epitope as nivolumab.

In some aspects, the anti-PD-1 antibody is a biosimilar of nivolumab.

In some aspects, the anti-PD-1 antibody is nivolumab.

In some aspects, the anti-PD-1 antibody cross-competes with pembrolizumab for binding to human PD-1.

In some aspects, the anti-PD-1 antibody binds to the same epitope as pembrolizumab.

In some aspects, the anti-PD-1 antibody is a biosimilar of pembrolizumab.

In some aspects, the anti-PD-1 antibody is pembrolizumab.

In some aspects, the anti-PD-1 antibody is nivolumab, pembrolizumab, PDR001, MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

### II.B.4.b Anti-PD-L1 Antibodies

In certain aspects, an anti-PD-L1 antibody is substituted for an anti-PD-1 antibody in any of the methods or uses disclosed herein.

Anti-PD-L1 antibodies that are known in the art can be used in the methods and uses of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31 (suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15supp/): TPS3113 (2018)), ICO 36, and CK-301 (Checkpoint Therapeutics; see Gorelik et al., AACR: Abstract 4606 (April 2016)).

Anti-PD-L1 antibodies that can be used in the methods and for the uses of the disclosure also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. In certain aspects, the antibodies that cross-compete for binding to human PD-L1 with, or bind to the same epitope region as, any anti-PD-L1 antibody disclosed herein, e.g., atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies that can be used in the methods and for the uses of the disclosure also include antigen-binding portions of any of the above full-length antibodies.

Anti-PD-L1 antibodies that can be used in the methods and for the uses of the disclosure are antibodies that bind to PD-L1 with high specificity and affinity, block the binding of PD-1, and inhibit the immunosuppressive effect of the PD-1 signaling pathway. In any of the methods or uses disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

Biosimilars of any of the anti-PD-L1 antibodies disclosed herein can also be used in the methods and for the uses of the disclosure.

In other aspects, the anti-PD-L1 antibody has the heavy and light chain CDRs or variable regions of any of the anti-PD-L1 antibodies disclosed herein, e.g., atezolizumab. Accordingly, in one aspect, the antibody comprises CDR1, CDR2, and CDR3 domains of the VH region of an anti-PD-L1 antibody disclosed herein, e.g., atezolizumab, and CDR1, CDR2 and CDR3 domains of the VL region of the antibody, e.g., atezolizumab. In another aspect, the anti-PD-L1 antibody comprises VH and/or VL regions of any of the anti-PD-L1 antibodies disclosed herein, e.g., atezolizumab.

In some aspects, the anti-PD-L1 antibody is a full-length antibody.

In some aspects, the anti-PD-L1 antibody is a monoclonal, chimeric, humanized, human, or multispecific antibody. In some aspects, the multispecific antibody is a dual-affinity re-targeting antibody (DART), a DVD-Ig, or bispecific antibody.

In some aspects, the anti-PD-L1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, CK-301, or comprises an antigen binding portion thereof.

### II.B.5. Anti-CTLA-4 Antibodies

In some aspects, the checkpoint inhibitor a disclosed herein comprises a CTLA-4 inhibitor. In some aspects, the CTLA-4 inhibitor is an anti-CTLA-4 antibody.

Anti-CTLA-4 antibodies that are known in the art can be used in the methods and uses of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)).

In some aspects, the anti-CTLA-4 antibody binds specifically to human CTLA-4 and cross-competes for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. In some aspects, the antibodies that cross-compete for binding to human CTLA-4 with, or bind to the same epitope region as, any anti-CTLA-4 antibody disclosed herein, e.g., ipilimumab and/or tremelimumab, are monoclonal antibodies. For administration to human subjects, these cross-competing antibodies are chimeric antibodies, engineered antibodies, or humanized or human antibodies.

Anti-CTLA-4 antibodies that can be used in the methods and for the uses of the disclosure also include antigen-binding portions of any of the above full-length antibodies.

Biosimilars of any of the anti-CTLA-4 antibodies disclosed herein can also be used in the methods and for the uses of the disclosure.

In other aspects, the anti-CTLA-4 antibody has the heavy and light chain CDRs or variable regions of any of the anti-CTLA-4 antibodies disclosed herein, e.g., ipilimumab or tremelimumab. Accordingly, in one aspect, the antibody comprises CDR1, CDR2, and CDR3 domains of the VH region of an anti-CTLA-4 antibody disclosed herein, e.g., ipilimumab or tremelimumab, and CDR1, CDR2 and CDR3 domains of the VL region of the antibody, e.g., ipilimumab or tremelimumab. In another aspect, the anti-CTLA-4 antibody comprises VH and/or VL regions of any of the anti-CTLA-4 antibodies disclosed herein, e.g., ipilimumab or tremelimumab.

In some aspects, the anti-CTLA-4 antibody is a full-length antibody.

In some aspects, the anti-CTLA-4 antibody is a monoclonal, human, humanized, chimeric, or multispecific antibody. In some aspects, the multispecific antibody is a DART, a DVD-Ig, or bispecific antibody.

In some aspects, the anti-CTLA-4 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-CTLA-4 antibody is ipilimumab, tremelimumab, MK-1308, AGEN-1884, or comprises an antigen binding portion thereof.

### II.B.6. Anti-TIM-3 Antibodies

In some aspects, the checkpoint inhibitor a disclosed herein comprises a TIM-3 inhibitor. In some aspects, the TIM-3 inhibitor is an anti-TIM-3 antibody.

Anti-TIM-3 antibodies that are known in the art can be used in the presently described compositions and methods.

In some aspects, the anti-TIM-3 antibody is TSR-022, LY3321367, or an anti-TIM-3 antibody disclosed in WO 2018/013818, which is incorporated by reference in its entirety.

In some aspects, the anti-TIM-3 antibody binds specifically to human TIM-3 and cross-competes for binding to human TIM-3 with any anti-TIM-3 antibody disclosed herein. In some aspects, the anti-TIM-3 antibody binds the same epitope as any of the anti-TIM-3 antibodies described herein. In some aspects, the antibodies that cross-compete for binding to human TIM-3 with, or bind to the same epitope region as, any anti-TIM-3 antibody disclosed herein are monoclonal antibodies. For administration to human subjects, these cross-competing antibodies are chimeric antibodies, engineered antibodies, or humanized or human antibodies.

Anti-TIM-3 antibodies that can be used in the methods and for the uses of the disclosure also include antigen-binding portions of any of the above full-length antibodies.

Biosimilars of any of the anti-TIM-3 antibodies disclosed herein can also be used in the methods and for the uses of the disclosure.

In other aspects, the anti-TIM-3 antibody has the heavy and light chain CDRs or variable regions of any of the anti-TIM-3 antibodies disclosed herein. Accordingly, in one aspect, the antibody comprises CDR1, CDR2, and CDR3 domains of the VH region of an anti-TIM-3 antibody disclosed herein, and CDR1, CDR2 and CDR3 domains of the VL region of the antibody. In another aspect, the anti-TIM-3 comprises VH and/or VL regions of any of the anti-TIM-3 antibodies disclosed herein.

In some aspects, the anti-TIM-3 antibody is a full-length antibody.

In some aspects, the anti-TIM-3 antibody is a monoclonal, human, humanized, chimeric, or multispecific antibody. In some aspects, the multispecific antibody is a DART, a DVD-Ig, or bispecific antibody.

In some aspects, the anti-TIM-3 antibody is a F(ab′)₂ fragment, a Fab′ fragment, a Fab fragment, a Fv fragment, a scFv fragment, a dsFv fragment, a dAb fragment, or a single chain binding polypeptide.

### II.C. Cancers

In some aspects, a cancer as disclosed herein is selected from the group consisting of breast cancer, hepatocellular cancer, gastroesophageal cancer, melanoma, bladder cancer, gastric cancer, lung cancer, kidney cancer, head and neck cancer, colon cancer, and any combination thereof.

In some aspects, a tumor or tumor sample disclosed herein is associated with a cancer selected from the group consisting of breast cancer, hepatocellular cancer, gastroesophageal cancer, melanoma, bladder cancer, gastric cancer, lung cancer, kidney cancer, head and neck cancer, colon cancer, and any combination thereof.

In some aspects, the cancer is a bladder cancer. In some aspects, the cancer is a gastric cancer. In some aspects, the cancer is a melanoma. In some aspects, the cancer is a lung cancer. In some aspects, the cancer is a breast cancer. In some aspects, the cancer is a hepatocellular cancer.

Cancers and benign lesions that can be treated by the methods and uses as disclosed herein include, but are not limited to, cancers and benign lesions of the circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites, or a combination of one or more thereof.

### II.D. Tumor Mutational Burden (TMB) Status

In some aspects, the methods and uses as disclosed herein further comprise measuring a tumor mutational burden (TMB) status.

TMB is a genetic analysis of a tumor's genome and, thus, can be measured by applying sequencing methods well known to those of skill in the art. The tumor DNA can be compared with DNA from patient-matched normal tissue to eliminate germline mutations or polymorphisms.

In some aspects, TMB is determined by sequencing tumor DNA using a high-throughput sequence technique, e.g., next-generation sequencing (NGS) or an NGS-based method. In some aspects, the NGS-based method is selected from whole genome sequencing (WGS), whole exome sequencing (WES), or comprehensive genomic profiling (CGP) of cancer gene panels such as FOUNDATIONONE® CDX™ and MSK-IMPACT clinical tests. In some aspects, TMB, as used herein, refers to the number of somatic mutations per megabase (Mb) of DNA sequenced. In one aspect, TMB is measured using the total number of nonsynonymous mutations, e.g., missense mutation (i.e., changing a particular amino acid in the protein) and/or nonsense (causing premature termination and thus truncation of the protein sequence), identified by normalizing matched tumor with germline samples to exclude any inherited germline genetic alterations. In another aspect, TMB is measured using the total number of missense mutations in a tumor. In order to measure TMB, a sufficient amount of sample is required. In one aspect, tissue sample (for example, a minimum of 10 slides) is used for evaluation. In some aspects, TMB is expressed as NsMs per megabase (NsM/Mb). 1 megabase represents 1 million bases.

The TMB status can be a numerical value or a relative value, e.g., high, medium, or low; within the highest fractile, or within the top tertile, of a reference set.

In some aspects, the TMB status is a high TMB.

In some aspects, a “high TMB” refers to a number of somatic mutations in a tumor's genome that is above a number of somatic mutations that is normal or average. In some aspects, a high TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500. In some aspects, a high TMB status has a score of at least at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250. In some aspects, a high TMB status has a score of at least 243.

In some aspects, a “high TMB” refers to a TMB within the highest fractile of the reference TMB value. For example, all subjects with evaluable TMB data are grouped according to fractile distribution of TMB, i.e., subjects are rank ordered from highest to lowest number of genetic alterations and divided into a defined number of groups. In some aspects, all subjects with evaluable TMB data are rank ordered and divided into thirds and a “high TMB” is within the top tertile of the reference TMB value. In some aspects, the tertile boundaries are 0<100 genetic alterations; 100 to 243 genetic alterations; and >243 genetic alterations. It should be understood that, once rank ordered, subjects with evaluable TMB data can be divided into any number of groups, e.g., quartiles, quintiles, etc.

In some aspects, a “high TMB” refers to a TMB of at least about 20 mutations/tumor, at least about 25 mutations/tumor, at least about 30 mutations/tumor, at least about 35 mutations/tumor, at least about 40 mutations/tumor, at least about 45 mutations/tumor, at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, or at least about 100 mutations/tumor. In some aspects, a “high TMB” refers to a TMB of at least about 105 mutations/tumor, at least about 110 mutations/tumor, at least about 115 mutations/tumor, at least about 120 mutations/tumor, at least about 125 mutations/tumor, at least about 130 mutations/tumor, at least about 135 mutations/tumor, at least about 140 mutations/tumor, at least about 145 mutations/tumor, at least about 150 mutations/tumor, at least about 175 mutations/tumor, or at least about 200 mutations/tumor. In certain aspects, a tumor having a high TMB has at least about 100 mutations/tumor.

In some aspects, “high TMB” can also be referred to as the number of mutations per megabase of genome sequenced, e.g., as measured by a mutation assay, e.g., FOUNDATIONONE® CDX™ assay. In one aspect, the high TMB refers to at least about 9, at least about 10, at least about 11, at least 12, at least about 13, at least about 14, at least about 15, at least about 16, at least about 17, at least about 18, at least about 19, or at least about 20 mutations per megabase of genome as measured by a FOUNDATIONONE® CDX™ assay. In a particular aspect, the “high TMB” refers to at least 10 mutations per megabase of genome sequenced by a FOUNDATIONONE® CDX™ assay.

As used herein, the term “medium TMB” refers to a number of somatic mutations in a tumor's genome that is at or around a number of somatic mutations that is normal or average and the term “low TMB” refers to a number of somatic mutations in a tumor's genome that is below a number of somatic mutations that is normal or average. In a particular aspect, a “high TMB” has a score of at least 243, a “medium TMB” has a score of between 100 and 242, and a “low TMB” has a score of less than 100 (or between 0 and 100). The “medium or low TMB” refers to less than 9 mutations per megabase of genome sequenced, e.g., as measured by a FOUNDATIONONE® CDX™ assay.

Microsatellite instability is the condition of genetic hypermutability that results from impaired DNA mismatch repair (MMR). The presence of MSI represents phenotypic evidence that MMR is not functioning normally. In most cases, the genetic basis for instability in MSI tumors is an inherited germline alteration in any one of the five human MMR genes: MSH2, MLH1, MSH6, PMS2, and PMS1. In certain aspects, the subject receiving tumor treatment has a high degree of microsatellite instability (MSI-H) and has at least one mutation in genes MSH2, MLH1, MSH6, PMS2, or PMS1. In other aspects, subjects receiving tumor treatment within a control group have no microsatellite instability (MSS or MSI stable) and has no mutation in genes MSH2, MLH1, MSH6, PMS2, and PMS1.

### II.E. PD-L1 Expression in the Tumor

In some aspects, the methods and uses as disclosed herein further comprise measuring membranous PD-L1 expression in a tumor sample obtained from the subject.

In some aspects, membranous PD-L1 expression in the tumor is assayed by immunohistochemistry (IHC), e.g., with the mAb 28-8.

In some aspects, the tumor is PD-L1 positive.

In some aspects, a PD-L1 positive tumor or PD-L1 expression positive tumor means that at least about 0.01%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, or at least about 30% of the total number of cells express PD-L1. PD-L1 positive tumor or PD-L1 expression positive tumor can also be referred to herein as tumor expressing PD-L1. In other aspects, the PD-L1 positive tumor or PD-L1 expression positive tumor means that at least about 0.1% to at least about 20% of the total number of cells express PD-L1. In certain aspects, the PD-L1 positive tumor or PD-L1 expression positive tumor means that at least about 0.1% to at least about 10% of the total number of cells express PD-L1. In some aspects, the PD-L1 positive or PD-L1 expression positive tumor means that at least about 1% of the total number of cells express PD-L1 on the cell surface. In other aspects, the PD-L1 positive or PD-L1 expression positive tumor means that at least about 5% of the total number of cells express PD-L1 on the cell surface. In one particular aspect, PD-L1 positive or PD-L1 expression positive tumor means that at least about 1%, or in the range of 1-5% of the total number of cells express PD-L1 on the cell surface.

### II.F. LAG-3 Expression in the Tumor

In some aspects, the methods and uses as disclosed herein further comprise measuring LAG-3 expression in a tumor sample obtained from the subject.

In some aspects, LAG-3 expression in the tumor is assayed by immunohistochemistry (IHC).

In some aspects, the tumor is LAG-3 positive.

In some aspects, a LAG-3 positive tumor or a LAG-3 expression positive tumor means that at least about 0.01%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 40%, at least about 50%, at least about 60%, at least about 70%, at least about 80%, at least about 90%, or about 100% of the total number of cells express LAG-3. In other aspects, for LAG-3 expression assayed by immunohistochemistry (IHC) or flow cytometry, the LAG-3 positive tumor or LAG-3 expression positive tumor means that at least about 0.01%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 40%, at least about 50%, at least about 60%, at least about 70%, at least about 80%, at least about 90%, or about 100% of the total number of tumor-associated inflammatory cells (e.g., T cells, CD8+ T cells, CD4+ T cells, FOXP3+ cells, NK cells) express LAG-3. LAG-3 positive tumor or LAG-3 expression positive tumor can also be expressed herein as tumor expressing LAG-3. In some aspects, the LAG-3 positive tumor or LAG-3 expression positive tumor means that at least about 0.1% to at least about 20% of the total number of cells express LAG-3. In some aspects, a LAG-3 positive tumor or LAG-3 expression positive tumor means that at least about 0.1% to at least about 20% of the total number of tumor-associated inflammatory cells (e.g., T cells, CD8+ T cells, CD4+ T cells, FOXP3+ cells, NK cells) express LAG-3. In certain aspects, a LAG-3 positive tumor or LAG-3 expression positive tumor means that at least about 0.1% to at least about 10% of the total number of cells express LAG-3. In certain aspects, a LAG-3 positive tumor or LAG-3 expression positive tumor means that at least about 0.1% to at least about 10% of the total number of tumor-infiltrating inflammatory cells (e.g., T cells, CD8+ T cells, CD4+ T cells, FOXP3+ cells, NK cells) express LAG-3. In some aspects, a LAG-3 positive or LAG-3 expression positive tumor means that at least about 1% of the total number of cells express LAG-3 on the cell surface. In some aspects, a LAG-3 positive or LAG-3 expression positive tumor means that at least about 1% of the total number of tumor-infiltrating inflammatory cells (e.g., T cells, CD8+ T cells, CD4+ T cells, FOXP3+ cells, NK cells) express LAG-3 on the cell surface. In other aspects, a LAG-3 positive or LAG-3 expression positive tumor means that at least about 5% of the total number of cells express LAG-3 on the cell surface. In other aspects, a LAG-3 positive or LAG-3 expression positive tumor means that at least about 5% of the total number of tumor-infiltrating inflammatory cells (e.g., T cells, CD8+ T cells, CD4+ T cells, FOXP3+ cells, NK cells) express LAG-3 on the cell surface. In one particular aspect, LAG-3 positive or LAG-3 expression positive tumor means that at least about 1%, or in the range of 1-5% of the total number of cells express LAG-3 on the cell surface. In one particular aspect, LAG-3 positive or LAG-3 expression positive tumor means that at least about 1%, or in the range of 1-5% of the total number of tumor-infiltrating inflammatory cells (e.g., T cells, CD8+ T cells, CD4+ T cells, FOXP3+ cells, NK cells) express LAG-3 on the cell surface.

### II.G. Treatment Protocols

In some aspects, suitable treatment protocols for treating a cancer in a human subject include administering to the patient an effective amount of a LAG-3 antagonist as disclosed herein (e.g., an anti-LAG-3 antibody such as relatlimab) or administration of an effective amount of a LAG-3 antagonist as disclosed herein (e.g., an anti-LAG-3 antibody such as relatlimab) and administration of an effective amount of a checkpoint inhibitor as disclosed herein (e.g., an anti-PD-1 antibody such as nivolumab).

In some aspects, the LAG-3 antagonist and/or the checkpoint inhibitor is administered at a weight-based dose.

In some aspects, the LAG-3 antagonist and/or the checkpoint inhibitor is administered at a flat dose.

In some aspects the LAG-3 antagonist and/or the checkpoint inhibitor is formulated for intravenous administration.

In some aspects, the LAG-3 antagonist and the checkpoint inhibitor are formulated separately. In some aspects, each checkpoint inhibitor is formulated separately when the checkpoint inhibitor comprises more than one checkpoint inhibitor. In some aspects, the checkpoint inhibitor is administered before the LAG-3 antagonist. In some aspects, the LAG-3 antagonist is administered before the checkpoint inhibitor.

In some aspects, the LAG-3 antagonist and the checkpoint inhibitor are formulated together (i.e., as a single composition).

In some aspects, two or more checkpoint inhibitors are formulated together when the checkpoint inhibitor comprises more than one checkpoint inhibitor.

In some aspects, the LAG-3 antagonist and the checkpoint inhibitor are administered concurrently.

In some aspects, the anti-LAG-3 antagonist is an anti-LAG-3 antibody (e.g., relatlimab) and the checkpoint inhibitor is an anti-PD-1 antibody (e.g., nivolumab).

In some aspects, the anti-LAG-3 antibody is administered at a dose from about 0.0001 to about 100 mg/kg or about 0.01 to about 5 mg/kg of the subject's body weight. For example doses can be about 0.3 mg kg body weight, about 1 mg/kg body weight, about 3 mg/kg body weight, about 5 mg/kg body weight or about 10 mg/kg body weight or within the range of about 1 to about 10 mg/kg. In some aspects, the anti-LAG-3 antibody is administered once per week, once every two weeks, once every three weeks, once every four weeks, once a month, once every 3 months or once every three to 6 months. In some aspects, an anti-LAG-3 antibody is administered at about 1 mg/kg body weight or about 3 mg/kg body weight via intravenous administration, with the antibody being given using one of the following dosing schedules: (i) every four weeks for six doses, then every three months; (ii) every three weeks; (iii) 3 mg/kg body weight once followed by 1 mg/kg body weight every three weeks. In some methods, the dose is adjusted to achieve a plasma antibody concentration of about 1-1000 μg/ml and in some methods about 25-300 μg/ml.

In some aspects, an anti-LAG-3 antibody or a combination of an anti-LAG-3 antibody and an anti-PD-1 antibody, or an anti-PD-L1 antibody, is administered at a dose of about 0.1, about 0.5, about 1, about 2, about 3, about 4, about 5, about 10, about 15, about 20, about 50, about 75, about 80, about 200, about 240, about 300, about 360, about 400, about 480, about 500, about 750 or about 1,500 mg of antibody.

In some aspects, the dose of an anti-LAG-3 antibody is administered every one week, every two weeks, every three weeks, every four weeks, every five weeks, every six weeks, every seven weeks, every eight weeks, every nine weeks, every ten weeks, every eleven weeks, or every twelve weeks.

In some aspects, the anti-LAG-3 antibody is administered at a dose of about 1, about 3, about 10, about 20, about 50, about 80, about 100, about 120, about 130, about 150, about 160, about 180, about 200, about 240 or about 280 mg and the anti-PD-1 antibody is administered at a dose of about 50, about 80, about 100, about 130, about 150, about 180, about 200, about 240, about 280, about 320, about 360, about 400, about 440, or about 480 mg. In some aspects, the anti-LAG-3 antibody is administered at a dose of about 320, about 360, about 400, about 440, about 480, about 520, about 560, about 600, about 640, about 680, about 720, about 760, about 800, about 840, about 880, about 920, about 960, or about 1000 mg. In some aspects, the anti-LAG-3 antibody is administered at a dose of about 1040, about 1080, about 1120, about 1160, about 1200, about 1240, about 1280, about 1320, about 1360, about 1400, about 1440, about 1480, about 1520, about 1560, about 1600, about 1640, about 1680, about 1720, about 1760, about 1800, about 1840, about 1880, about 1920, about 1960, or about 2000 mg. In some aspects, the anti-LAG-3 antibody is administered at a dose of about 480 mg. In some aspects, the anti-LAG-3 antibody is administered at a dose of about 0.01, about 0.03, about 0.25, about 0.1, about 0.3, about 1, about 3, about 5, about 8 or about 10 mg/kg body weight and the anti-PD-1 antibody is administered at a dose of about 0.1, about 0.3, about 1, about 3, about 5, about 8 or about 10 mg/kg body weight.

In some aspects, the anti-LAG-3 antibody and anti-PD-1 antibody are administered at about 80 mg anti-LAG-3 antibody and about 240 mg of anti-PD-1 antibody.

In some aspects, the anti-LAG-3 antibody and anti-PD-1 antibody are administered at about 160 mg anti-LAG-3 antibody and about 480 mg of anti-PD-1 antibody.

In some aspects, the anti-PD-1 antibody is nivolumab and is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 3 weeks. In some aspects, nivolumab is administered at a flat dose of about 480 mg once about every 4 weeks.

In some aspects, the checkpoint inhibitor is pembrolizumab and is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 4 weeks.

In some aspects, the checkpoint inhibitor is atezolizumab and is administered as a flat dose of about 800 mg once about every 2 weeks. In some aspects, atezolizumab is administered as a flat dose of about 840 mg once about every 2 weeks.

In some aspects, the checkpoint inhibitor is durvalumab and is administered at a dose of about 10 mg/kg once about every 2 weeks. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the checkpoint inhibitor is avelumab and is administered as a flat dose of about 800 mg once about every 2 weeks.

In some aspects, the checkpoint inhibitor is ipilimumab and is administered at a dose of at least about 3 mg/kg once about every 3 weeks. In some aspects, ipilimumab is administered at a dose of at least about 10 mg/kg once about every 3 weeks. In some aspects, ipilimumab is administered at a dose of at least about 10 mg/kg once about every 12 weeks. In some aspects, the ipilimumab is administered for four doses.

### II.H. Outcomes

Patients treated according to the methods and uses disclosed herein preferably experience improvement in at least one sign of cancer. In one aspect, improvement is measured by a reduction in the quantity and/or size of measurable tumor lesions. In another aspect, lesions can be measured on chest x-rays or CT or MRI films. In another aspect, cytology or histology can be used to evaluate responsiveness to a therapy.

In one aspect, the patient treated exhibits a complete response (CR), a partial response (PR), stable disease (SD), immune-related complete disease (irCR), immune-related partial response (irPR), or immune-related stable disease (irSD). In another aspect, the patient treated experiences tumor shrinkage and/or decrease in growth rate, i.e., suppression of tumor growth. In another aspect, unwanted cell proliferation is reduced or inhibited. In yet another aspect, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other aspects, the methods and uses provided herein produces at least one therapeutic effect selected from the group consisting of reduction in size of a tumor, reduction in number of metastatic lesions appearing over time, complete remission, partial remission, or stable disease.

In still other aspects, the methods and uses provided herein produce an objective response rate (ORR=CR+PR) of at least about 15%, at least about 20%, at least about 25%, at least about 30%, at least about 40%, at least about 50%, at least about 60%, at least about 70%, at least about 80%, at least about 90%, or about 100%. In some aspects, the median duration of response is ≥3 month, ≥6 month, ≥12 month, or ≥18 month. In one aspect, the median duration of response is ≥6 month. In some aspects, the frequency of patients with duration of response ≥6 month is at least about 30%, at least about 40%, at least about 50%, at least about 60%, at least about 70%, at least about 80%, at least about 90%, at least about 95%, at least about 99%, or about 100%.

In still other aspects, the methods and uses provided herein produce a disease control rate (DRR=CR+PR+SD) of at least about 20%, at least about 30%, at least about 40%, at least about 50%, at least about 60%, at least about 70%, at least about 80%, at least about 90%, at least about 95%, at least about 99% or about 100%. In some aspects, the median duration of response is ≥3 month, ≥6 month, ≥12 month, or ≥18 month. In one aspect, the median duration of response is ≥6 month. In some aspects, the frequency of patients with duration of response ≥6 month is at least about 30%, at least about 40%, at least about 50%, at least about 60%, at least about 70%, at least about 80%, at least about 90%, at least about 95%, at least about 99% or 100%.

In some aspects, the subject exhibits improved overall survival or progression free survival compared to a non-responder (a subject with a low LAG-3 D score, a low LAG-3-P score, or both).

In some aspects, the administering treats the cancer.

In some aspects, the administering reduces the size of a tumor associated with the cancer.

In some aspects, the size of the tumor is reduced by at least about 10%, about 20%, about 30%, about 40%, or about 50% compared to the tumor size prior to the administration.

In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some aspects, the subject exhibits stable disease after the administration.

In some aspects, the subject exhibits a partial response after the administration.

In some aspects, the subject exhibits a complete response after the administration.

### III. KITS

Also within the scope of the present disclosure are kits comprising (a) a dosage of a LAG-3 antagonist as disclosed herein, including any of the doses disclosed herein.

Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage of the LAG-3 antagonist, including any of the doses disclosed herein; and (b) instructions for using the LAG-3 antagonist in the methods and uses disclosed herein.

In certain aspects for treating human patients, the kit further comprises an PD-1 pathway inhibitor. In some aspects, the kit comprises a dosage the PD-1 pathway inhibitor, including any of the doses for checkpoint inhibitors disclosed herein. In some aspects, the PD-1 pathway inhibitor is an anti-human PD-1 antibody disclosed herein, e.g., nivolumab or pembrolizumab, and/or an anti-PD-L1 antibody disclosed herein, e.g., atezolizumab, durvalumab, or avelumab. In some aspects, the kit comprises a dosage of an anti-PD-1 antibody and/or an anti-PD-L1 antibody.

In some aspects, the kit further comprises an anti-CTLA-4 antibody and/or an anti-TIM-3 antibody.

All of the references cited above, as well as all references cited herein, are incorporated herein by reference in their entireties.

The following examples are offered by way of illustration and not by way of limitation.

### EXAMPLES

### Example 1

Based on MHC II ligand engagement of the LAG-3 receptor, MHC II and LAG-3 interactions were investigated in exemplary tumor samples with quantitative spatial profiling, which utilizes serial immunohistochemistry (IHC)-stained slide sections to define the geographic distribution of markers, both individually and in relation to one another.

### Hypothesis and Objective

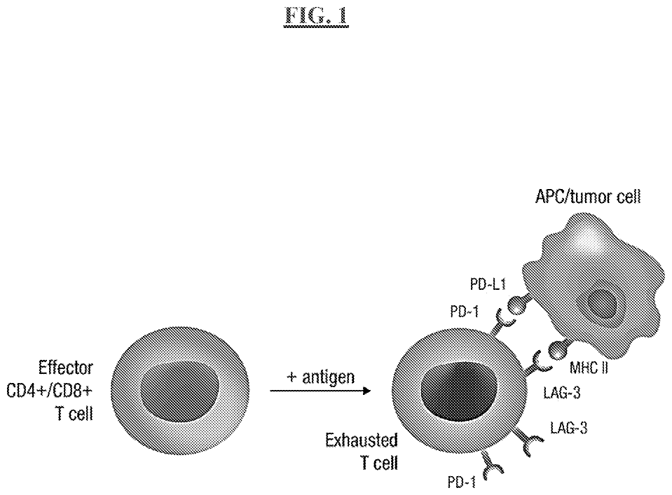

It was hypothesized that localization of MHC II ligand engagement of the LAG-3 receptor within a specified proximity will allow for engagement and activation of the LAG-3 checkpoint and T-cell exhaustion (FIG. 1).

Thus, an objective of this study was to characterize the spatial association of LAG-3+ tumor infiltrating lymphocytes (TILs) with individual tumor cells that are either MHC II+ or MHC II− using digital spatial analysis.

### Methods

Commercially procured gastric and bladder tumor samples (n=20 of each tumor type) were obtained.

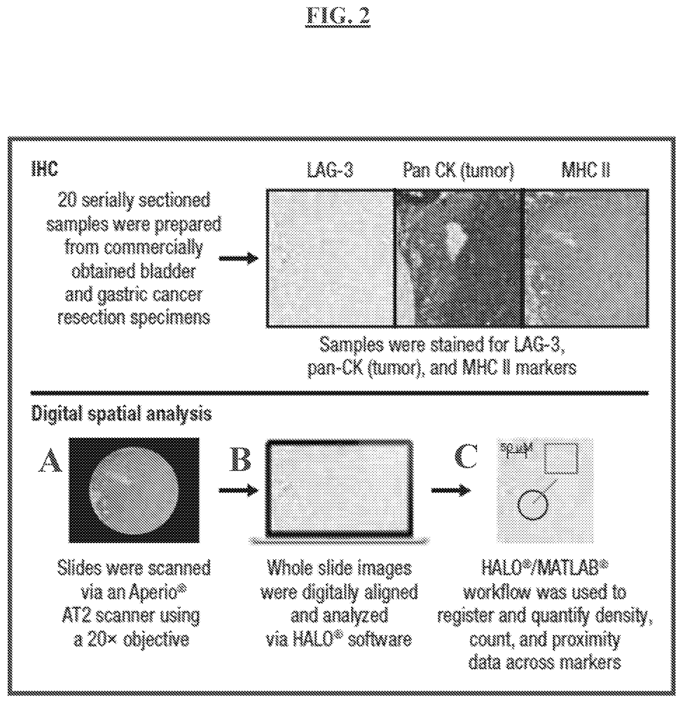

Tumor samples were serially sectioned and stained by IHC (FIG. 2) for: (1) LAG-3: using a monoclonal antibody directed against 17b4, (2) Pan cytokeratin (Pan CK): using a monoclonal antibody directed against AE1-AE3 to identify epithelial cell lineage in tumors, and (3) MHC II: using a monoclonal antibody directed against CR3/43.

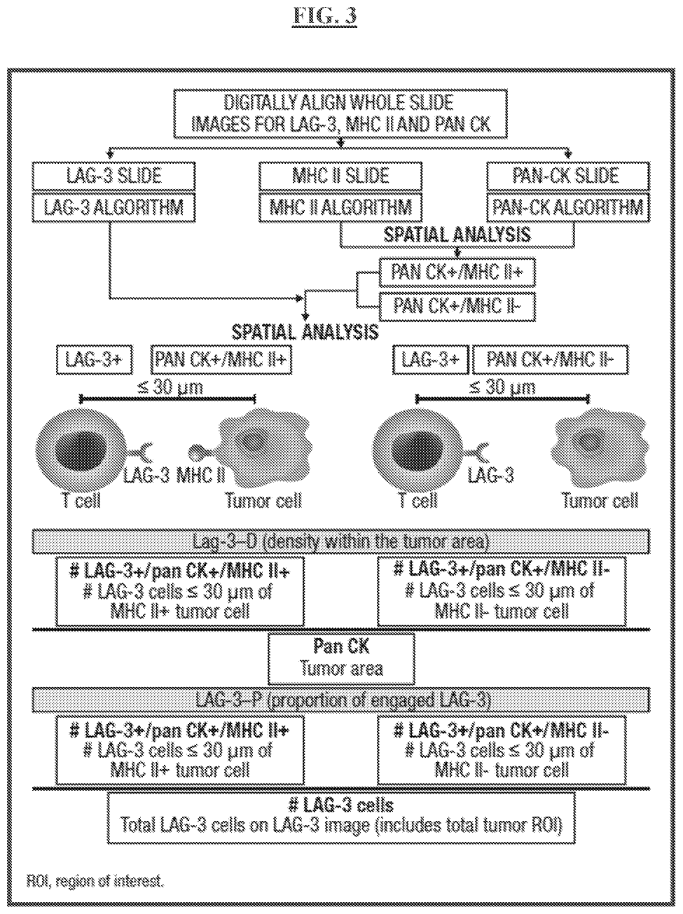

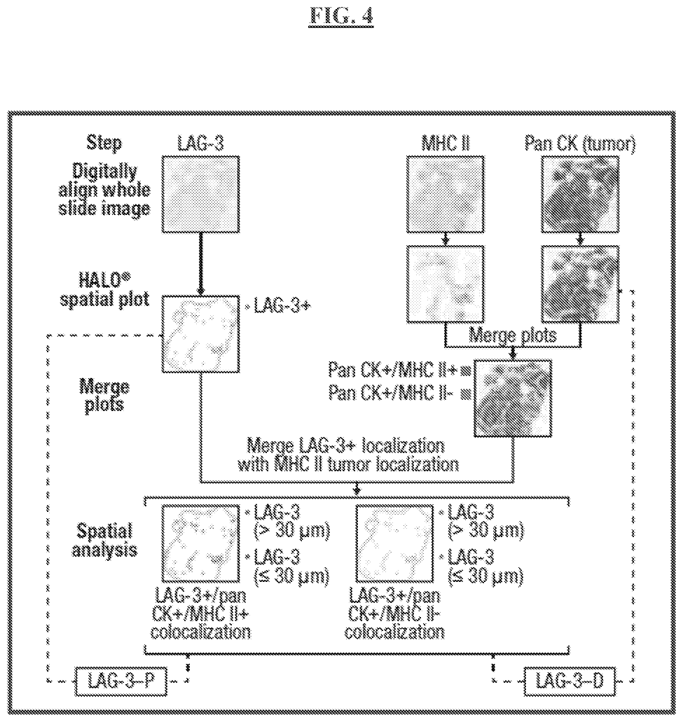

Digital spatial profiling was performed using the stained slides (FIGS. 2-4).

Briefly, slides were scanned with an APERIO® AT2 scanner using a 20× objective.

Whole slide images for LAG-3, MHC II, and Pan CK were digitally aligned and analyzed via HALO® software using respective algorithms for LAG-3, MHC II, and Pan CK to produce HALO® spatial plots that were merged for spatial analysis.

MHC II and Pan CK (tumor) plots were merged to identify MHC II+ and MHC-tumor cells. The LAG-3+ plots and Pan CK+/MHC II plots were then merged to determine the number of LAG-3+ TILs located ≤ or >30 μm of Pan CK+/MHC II+ or Pan CK+/MHC− tumor cells.

The HALO®/MATLAB® workflow was used to register and quantify density, count, and proximity data across markers. LAG-3 engagement scores representing the density (D) of LAG-3+ TILs within ≤30 μm of MHC II+vs MHC II− tumor cells (i.e., LAG-3-D) and the proportion (P) of LAG-3+ TILs within ≤30 μm of WIC II+vs WIC II− tumor cells (i.e., LAG-3-P) were computed for each sample using R software. LAG-3-D (cells/mm²) was calculated as the number of LAG-3+ TILs within ≤30 μm of MHC II+ or MHC II− tumor cells divided by the pan CK+ tumor area. LAG-3-P (%) was calculated as the number of LAG-3+ TILs within ≤30 μm of WIC II+ or WIC II− tumor cells divided by the total number of LAG-3+ cells on the LAG-3 image, which includes the total tumor region of interest (ROI). A Mann-Whitney test was conducted to assess statistical differences between the proportion of LAG-3+ TILs within ≤30 μm to WIC II+ and MHC− II tumor cells.

### Results

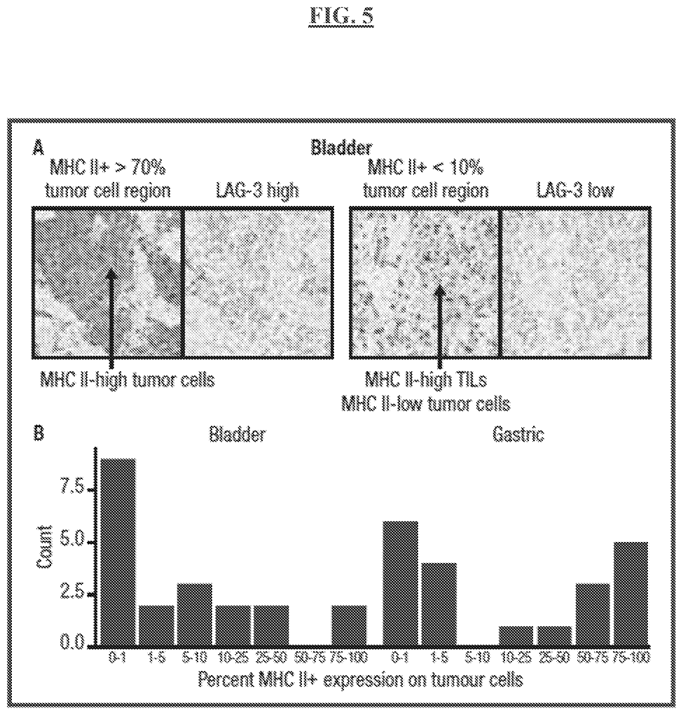

In the samples investigated for this analysis, there was a dynamic range of expression of MHC II (FIG. 5). MHC II was expressed by at least 1% of tumor cells in 55% of bladder and 70% of gastric samples.

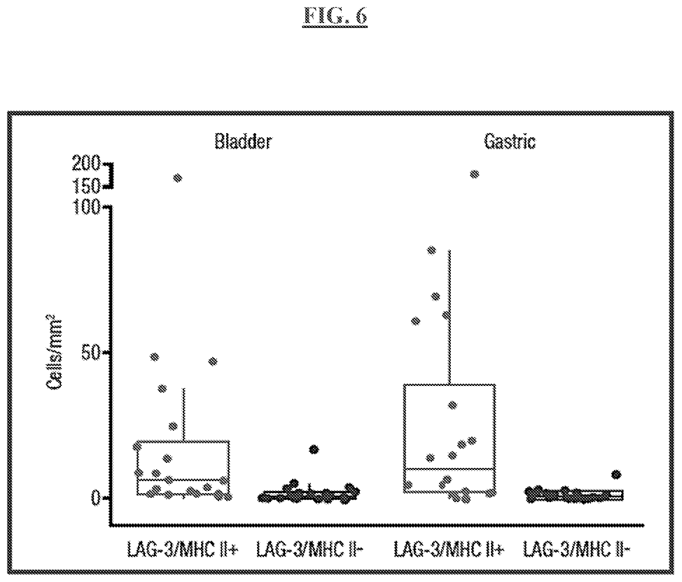

In both bladder and gastric cancers, the density of LAG-3 engagement was higher in MHC II+ tumor regions (median [interquartile range] 6.53 [1.76, 24.9] cells/mm²) compared to MHC II− tumor cells (0.616 [0.213, 2.38] cells/mm² [P<0.001]) (FIG. 6).

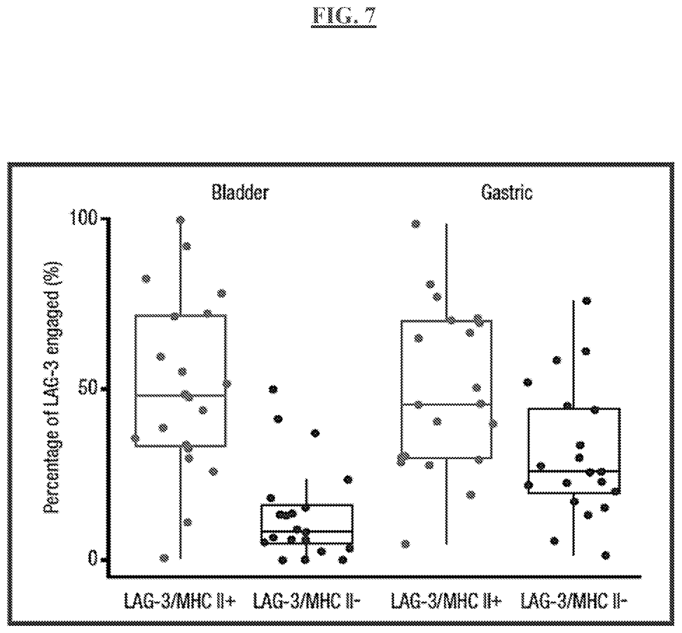

The proportion of LAG-3+ TILs within ≤30 μm of tumor cells was higher in MHC II+ tumor cells (median [interquartile range] 46.7 [30.1, 70.4] % engaged) compared to LAG-3+ TILs within 30 μm of MHC II− tumor cells (17.5 [6.09, 30.1] % engaged [P<0.001]) in both bladder and gastric cancers (FIG. 7).

### CONCLUSIONS

These data suggest preferential localization of LAG-3-expressing TILs to MHC II+ tumor cells within a proximity that may allow engagement and activation of the LAG-3 checkpoint, contributing to T-cell exhaustion.

Quantitative spatial analysis of tumor cells and TILs in the tumor microenvironment was feasible and captured cell-cell relationships in tumors with heterogeneous MHC II expression.

What is claimed is:

1. A method of treating a cancer in a human subject in need thereof comprising administering a lymphocyte activation gene-3 (LAG-3) antagonist to the subject, wherein the subject is identified as having (i) a high LAG-3 density (LAG-3-D) score, (ii) a high LAG-3 proportion (LAG-3-P) score, or (iii) both a high LAG-3-D score and a high LAG-3-P score in a tumor sample obtained from the subject, wherein the LAG-3-D score is determined by measuring a density of T cells expressing LAG-3 in proximity to one or more tumor cells expressing major histocompatibility complex class II (MHC II) in the tumor sample, and wherein the LAG-3-P score is determined by measuring a proportion of T cells expressing LAG-3 in proximity to one or more tumor cells expressing MHC II in the tumor sample.

2. A method of treating a cancer in a human subject in need thereof comprising (a) identifying a subject having (i) a high LAG-3-D score, (ii) a high LAG-3-P score, or (iii) both a high LAG-3-D score and a high LAG-3-P score in a tumor sample obtained from the subject and (b) administering a LAG-3 antagonist to the subject, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample.

3. A method of identifying a human subject afflicted with a cancer suitable for a LAG-3 antagonist therapy, comprising computing (i) a LAG-3-D score, (ii) a LAG-3-P score, or (iii) both a LAG-3-D score and a LAG-3-P score in a tumor sample obtained from a subject in need of a LAG-3 antagonist therapy, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample.

4. The method of claim 3, wherein the subject exhibits a high LAG-3-D score, a high LAG-3-P score, or both a high LAG-3-D score and a high LAG-3-P score.

5. The method of claim 3 or 4, further comprising administering a LAG-3 antagonist to the subject.

6. A LAG-3 antagonist for treating a cancer in a human subject in need thereof, wherein the subject is identified as having (i) a high LAG-3-D score, (ii) a high LAG-3-P score, or (iii) both a high LAG-3-D score and a high LAG-3-P score of a tumor sample obtained from the subject, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample.

7. A LAG-3 antagonist for identifying a subject afflicted with a cancer suitable for a LAG-3 antagonist therapy, wherein (i) a LAG-3-D score, (ii) a LAG-3-P score, or (iii) both a LAG-3-D score and a LAG-3-P score in a tumor sample obtained from the subject is computed, wherein the LAG-3-D score is determined by measuring a density of T cells expressing LAG-3 in proximity to one or more tumor cells expressing MHC II in the tumor sample, and wherein the LAG-3-P score is determined by measuring a proportion of T cells expressing LAG-3 in proximity to one or more tumor cells expressing MHC II in the tumor sample.

8. The LAG-3 antagonist for use of claim 7, wherein the subject exhibits a high LAG-3-D score, a high LAG-3-P score, or both a high LAG-3-D score and a high LAG-3-P score.

9. The method of any one of claims 1 to 5 or the LAG-3 antagonist for use of any one of claims 6 to 8, wherein the LAG-3-D score is calculated as (i) the number of the T cells expressing LAG-3 in proximity to the tumor cells expressing MHC II divided by (ii) the tumor area (mm²) of the tumor sample.

10. The method of any one of claims 1 to 5 and 9 or the LAG-3 antagonist for use of any one of claims 6 to 9, wherein the LAG-3-P score is calculated as (i) the number of the T cells expressing LAG-3 in proximity to the tumor cell expressing MHC II divided by (ii) the total number of T cells expressing LAG-3 in the tumor sample.

11. The method of any one of claims 1 to 5 and 9 and 10 or the LAG-3 antagonist for use of any one of claims 6 to 10, wherein the proximity is between the LAG-3 and the MHC class II and/or between the LAG-3 and a tumor antigen expressed on the tumor cells.

12. The method of any one of claims 1 to 5 and 9 to 11 or the LAG-3 antagonist for use of any one of claims 6 to 11, wherein the proximity is equal to or less than about 50 μm, equal to or less than about 45 μm, equal to or less than about 40 μm, equal to or less than about 35 μm, or equal to or less than about 30 μm.

13. The method of any one of claims 1 to 5 and 9 to 12 or the LAG-3 antagonist for use of any one of claims 6 to 12, wherein the proximity is equal to or less than about 30 μm.

14. The method of any one of claims 1 to 5 and 9 to 13 or the LAG-3 antagonist for use of any one of claims 6 to 13, wherein the tumor sample comprises one or more tumor sections derived from a tumor tissue biopsy or a tumor tissue resection.

15. The method or the LAG-3 antagonist for use of claim 14, wherein the one or more tumor sections comprise a formalin-fixed, paraffin-embedded tumor tissue or a fresh-frozen tumor tissue.

16. The method of or the LAG-3 antagonist for use of claim 14 or 15, wherein the one or more tumor sections comprise serially sectioned tumor sections.

17. The method or the LAG-3 antagonist for use of any one of claims 14 to 16, wherein the one or more tumor sections are stained by immunohistochemistry (IHC).

18. The method or the LAG-3 antagonist for use of any one of claims 14 to 17, wherein the one or more tumor sections comprise one tumor section, two tumor sections, three tumor sections, four tumor sections, five tumor sections, six tumor sections, seven tumor sections, eight tumor sections, nine tumor sections, ten tumor sections, 11 tumor sections, 12 tumor sections, 13 tumor sections, 14 tumor sections, 15 tumor sections, 16 tumor sections, 17 tumor sections, 18 tumor sections, 19 tumor sections, 20 tumor sections, 21 tumor sections, 22 tumor sections, 23 tumor sections, 24 tumor sections, 25 tumor sections, 26 tumor sections, 27 tumor sections, 28 tumor sections, 29 tumor sections, or 30 tumor sections.

19. The method of or the LAG-3 antagonist for use of any one of claims 14 to 18, wherein one tumor section of the tumor sample is stained for the LAG-3 and the MHC II.

20. The method or LAG-3 antagonist for use of claim 19, wherein the tumor section is further stained for a tumor antigen.

21. The method or LAG-3 antagonist for use of claim 20, wherein the tumor antigen is Pan cytokeratin (CK).

22. The method or LAG-3 antagonist for use of any one of claims 14 to 21, wherein the tumor sample comprises a first tumor section stained for the LAG-3, a second tumor section stained for the MHC II, and a third tumor section stained for a tumor antigen.

23. The method or LAG-3 antagonist for use of claim 22, wherein the first tumor section, the second tumor section, and the third tumor section are serially sectioned from the tumor sample.

24. The method of any one of claims 1 to 5 and 9 to 23 or the LAG-3 antagonist for use of any one of claims 6 to 23, wherein the high LAG-3-D score is at least about 5 cells/mm², at least about 10 cells/mm², at least about 15 cells/mm², at least about 20 cells/mm², at least about 25 cells/mm², at least about 30 cells/mm², at least about 35 cells/mm², at least about 40 cells/mm², at least about 45 cells/mm², at least about 50 cells/mm², at least about 55 cells/mm², at least about 60 cells/mm², at least about 65 cells/mm², at least about 70 cells/mm², at least about 75 cells/mm², at least about 80 cells/mm², at least about 85 cells/mm², at least about 90 cells/mm², at least about 95 cells/mm², or at least about 100 cell s/mm².

25. The method of any one of claims 1 to 5 and 9 to 24 or the LAG-3 antagonist for use of any one of claims 6 to 24, wherein the high LAG-3-P score is at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

26. The method of any one of claims 1 to 5 and 9 to 25 or the LAG-3 antagonist for use of any one of claims 6 to 25, wherein the subject exhibits improved overall survival or progression free survival compared to a non-responder (a subject with a low LAG-3 D score, a low LAG-3-P score, or both).

27. The method of any one of claims 1 to 5 and 9 to 26 or LAG-3 antagonist for use of any one of claims 6 to 26, further comprising measuring a tumor mutational burden (TMB) status.

28. The method of any one of claims 1 to 5 and 9 to 27 or the LAG-3 antagonist for use of any one of claims 6 to 27, wherein the subject exhibits a high TMB.

29. The method of any one of claims 1 to 5 and 9 to 28 or the LAG-3 antagonist for use of any one of claims 6 to 28, further comprising measuring membranous PD-L1 expression in the tumor.

30. The method or LAG-3 antagonist for use of claim 29, wherein the tumor is PD-L1 positive.

31. The method of any one of claims 1 to 5 and 9 to 30 or the LAG-3 antagonist for use of any one of claims 6 to 30, wherein the tumor is LAG-3 positive.

32. The method of any one of claims 1 to 5 and 9 to 31 or the LAG-3 antagonist for use of any one of claims 6 to 31, wherein the LAG-3 antagonist is a soluble LAG-3 polypeptide.

33. The method or LAG-3 antagonist for use of claim 32, wherein the soluble LAG-3 polypeptide is a fusion polypeptide.

34. The method or LAG-3 antagonist for use of claim 32 or 33, wherein the soluble LAG-3 polypeptide comprises a ligand binding fragment of the LAG-3 extracellular domain.

35. The method or LAG-3 antagonist for use of any one of claims 32 to 34, wherein the soluble LAG-3 polypeptide further comprises a half-life extending moiety.

36. The method or LAG-3 antagonist for use of claim 35, wherein the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof.

37. The method or LAG-3 antagonist for use of any one of claims 32 to 36, wherein the soluble LAG-3 polypeptide is IMP321 (eftilagimod alpha).

38. The method of any one of claims 1 to 5 and 9 to 31 or the LAG-3 antagonist for use of any one of claims 6 to 31, wherein the LAG-3 antagonist is an anti-LAG-3 antibody.

39. The method or LAG-3 antagonist for use of claim 38, wherein the anti-LAG-3 antibody is a full-length antibody.

40. The method or LAG-3 antagonist for use of claim 38 or 39, wherein the anti-LAG-3 antibody is a monoclonal, chimeric, humanized, human, or multispecific antibody.

41. The method or LAG-3 antagonist for use of claim 40, wherein the multispecific antibody is a dual-affinity re-targeting antibody (DART), a DVD-Ig, or bispecific antibody.

42. The method or LAG-3 antagonist for use of claim 38, wherein the anti-LAG-3 antibody is a F(ab′)₂ fragment, a Fab′ fragment, a Fab fragment, a Fv fragment, a scFv fragment, a dsFv fragment, a dAb fragment, or a single chain binding polypeptide.

43. The method or LAG-3 antagonist for use of any one of claims 38 to 42, wherein the anti-LAG-3 antibody cross-competes with BMS-986016 (relatlimab) for binding to human LAG-3.

44. The method or LAG-3 antagonist for use of claim 38 or 43, wherein the anti-LAG-3 antibody binds to the same epitope as BMS-986016 (relatlimab).

45. The method or LAG-3 antagonist for use of any one of claims 38 to 42, wherein the anti-LAG-3 antibody is BMS-986016 (relatlimab), LAG-525 (IMP-701, ieramilimab), MK-4280 (28G-10), REGN3767 (fianlimab), TSR-033, TSR-075, Sym022, FS-118, IMP731 (H5L7BW), GSK2831781, humanized BAP050, aLAG3(0414), aLAG3(0416), XmAb22841, MGD013, B1754111, P 13B02-30, AVA-017, 25F7, AGEN1746, or comprises an antigen binding portion thereof.

46. The method of any one of claims 1 to 5 and 9 to 45 or the LAG-3 antagonist for use of any one of claims 6 to 45, wherein the LAG-3 antagonist is administered at a flat dose.

47. The method of any one of claims 1 to 5 and 9 to 45 or the LAG-3 antagonist for use of any one of claims 6 to 45, wherein the LAG-3 antagonist is administered at a weight-based dose.

48. The method or LAG-3 antagonist for use of claim 46 or 47, wherein the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

49. The method of any one of claims 1 to 5 and 9 to 48 or the LAG-3 antagonist for use of any one of claims 6 to 48, further comprising administering to the subject an additional therapeutic agent.

50. The method or LAG-3 antagonist for use of claim 49, wherein the additional therapeutic agent comprises an anti-cancer agent.

51. The method or LAG-3 antagonist for use of claim 50, wherein the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

52. The method or LAG-3 antagonist for use of claim 51, the tyrosine kinase inhibitor comprises sorafenib, lenvatinib, regorafenib, cabozantinib, sunitinib, brivanib, linifanib, erlotinib, pemigatinib, everolimus, gefitinib, imatinib, lapatinib, nilotinib, pazopanib, temsirolimus, or any combination thereof.

53. The method or LAG-3 antagonist for use of claim 51 or 52, wherein the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof.

54. The method or LAG-3 antagonist for use of any one of claims 51 to 53, wherein the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof.

55. The method or LAG-3 antagonist for use of any one of claims 51 to 54, wherein the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

56. The method or LAG-3 antagonist for use of any one of claims 51 to 55, wherein the checkpoint inhibitor comprises a PD-1 pathway inhibitor.

57. The method or LAG-3 antagonist for use of claim 55 or 56, wherein the PD-1 pathway inhibitor is an anti-PD-1 antibody and/or an anti-PD-L1 antibody.

58. The method or LAG-3 antagonist for use of claim 56 or 57, wherein the PD-1 pathway inhibitor is an anti-PD-1 antibody.

59. The method or LAG-3 antagonist for use of claim 57 or 58, wherein the anti-PD-1 antibody is a full-length antibody.

60. The method or LAG-3 antagonist for use of any one of claims 57 to 59, wherein the anti-PD-1 antibody is a monoclonal, chimeric, humanized, human, or multispecific antibody.

61. The method or LAG-3 antagonist for use of claim 60, wherein the multispecific antibody is a dual-affinity re-targeting antibody (DART), a DVD-Ig, or bispecific antibody.

62. The method or LAG-3 antagonist for use of claim 57 or 58, wherein the anti-PD-1 antibody is a F(ab′)2 fragment, a Fab′ fragment, a Fab fragment, a Fv fragment, a scFv fragment, a dsFv fragment, a dAb fragment, or a single chain binding polypeptide.

63. The method or LAG-3 antagonist for use of any one of claims 57 to 62, wherein the anti-PD-1 antibody cross-competes with nivolumab for binding to human PD-1.

64. The method or LAG-3 antagonist for use of any one of claims 57 to 63, wherein the anti-PD-1 antibody binds to the same epitope as nivolumab.

65. The method or LAG-3 antagonist for use of any one of claims 57 to 62, wherein the anti-PD-1 antibody cross-competes with pembrolizumab for binding to human PD-1.

66. The method or LAG-3 antagonist for use of any one of claim 57 to 62 or 65, wherein the anti-PD-1 antibody binds to the same epitope as pembrolizumab.

67. The method or LAG-3 antagonist for use of any one of claims 57 to 62, wherein the anti-PD-1 antibody is nivolumab, pembrolizumab, PDR001, MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

68. The method or LAG-3 antagonist for use of claim 55 or 56, wherein the PD-1 pathway inhibitor is a soluble PD-L2 polypeptide.

69. The method or LAG-3 antagonist for use of claim 68, wherein the soluble PD-L2 polypeptide is a fusion polypeptide.

70. The method or LAG-3 antagonist for use of claim 68 or 69, wherein the soluble PD-L2 polypeptide comprises a ligand binding fragment of the PD-L2 extracellular domain.

71. The method or LAG-3 antagonist for use of any one of claims 68 to 70, wherein the soluble PD-L2 polypeptide further comprises a half-life extending moiety.

72. The method or LAG-3 antagonist for use of claim 71, wherein the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof.

73. The method or LAG-3 antagonist for use of claim 72, wherein the soluble PD-L2 polypeptide is AMP-224.

74. The method or LAG-3 antagonist for use of claim 56 or 57, wherein the PD-1 pathway inhibitor is an anti-PD-L1 antibody.

75. The method or LAG-3 antagonist for use of claim 57 or 74, wherein the anti-PD-L1 antibody is a full-length antibody.

76. The method or LAG-3 antagonist for use of any one of claim 57, 74, or 75, wherein the anti-PD-L1 antibody is a monoclonal, chimeric, humanized, human, or multispecific antibody.

77. The method or LAG-3 antagonist for use of claim 76, wherein the multispecific antibody is a dual-affinity re-targeting antibody (DART), a DVD-Ig, or bispecific antibody.

78. The method or LAG-3 antagonist for use of claim 57 or 74, wherein the anti-PD-L1 antibody is a F(ab′)₂ fragment, a Fab′ fragment, a Fab fragment, a Fv fragment, a scFv fragment, a dsFv fragment, a dAb fragment, or a single chain binding polypeptide.

79. The method or LAG-3 antagonist for use of any one of claim 57 or 74 to 78, wherein the anti-PD-L1 antibody cross-competes with atezolizumab for binding to human PD-L1.

80. The method or LAG-3 antagonist for use of any one of claim 57 or 74 to 79, wherein the anti-PD-L1 antibody binds to the same epitope as atezolizumab.

81. The method or LAG-3 antagonist for use of any one of claim 57 or 74 to 78, wherein the anti-PD-L1 antibody cross-competes with durvalumab for binding to human PD-L1.

82. The method or LAG-3 antagonist for use of any one of claim 57, 74 to 78, or 81, wherein the anti-PD-L1 antibody binds to the same epitope as durvalumab.

83. The method or LAG-3 antagonist for use of any one of claim 57 or 74 to 78, wherein the anti-PD-L1 antibody cross-competes with avelumab for binding to human PD-L1.

84. The method or LAG-3 antagonist for use of any one of claim 57, 74 to 78, or 83, wherein the anti-PD-L1 antibody binds to the same epitope as avelumab.

85. The method or LAG-3 antagonist for use of any one of claim 57 or 74 to 78, wherein the anti-PD-L1 antibody is BMS-936559, atezolizumab, durvalumab, avelumab, STI-1014, CX-072, KN035, LY3300054, BGB-A333, ICO 36, CK-301, or comprises an antigen binding portion thereof.

86. The method or LAG-3 antagonist for use of claim 56 or 57, wherein the PD-1 pathway inhibitor is BMS-986189.

87. The method or LAG-3 antagonist for use of any one of claims 51 to 86, wherein the checkpoint inhibitor comprises a CTLA-4 inhibitor.

88. The method or LAG-3 antagonist for use of claim 87, wherein the CTLA-4 inhibitor is an anti-CTLA-4 antibody.

89. The method or LAG-3 antagonist for use of claim 88, wherein the anti-CTLA-4 antibody is a full-length antibody.

90. The method or LAG-3 antagonist for use of claim 88 or 89, wherein the anti-CTLA-4 antibody is a monoclonal, chimeric, humanized, human, or multispecific antibody.

91. The method or LAG-3 antagonist for use of claim 90, wherein the multispecific antibody is a dual-affinity re-targeting antibody (DART), a DVD-Ig, or bispecific antibody.

92. The method or LAG-3 antagonist for use of claim 88, wherein the anti-CTLA-4 antibody is a F(ab′)₂ fragment, a Fab′ fragment, a Fab fragment, a Fv fragment, a scFv fragment, a dsFv fragment, a dAb fragment, or a single chain binding polypeptide.

93. The method or LAG-3 antagonist for use of any one of claims 88 to 92, wherein the anti-CTLA-4 antibody cross-competes with ipilimumab for binding to human CTLA-4.

94. The method or LAG-3 antagonist for use of any one of claims 88 to 93, wherein the anti-CTLA-4 antibody binds to the same epitope as ipilimumab.

95. The method or LAG-3 antagonist for use of any one of claims 51 to 94, wherein the checkpoint inhibitor is formulated for intravenous administration.

96. The method or LAG-3 antagonist for use of any one of claims 51 to 95, wherein the LAG-3 antagonist and the checkpoint inhibitor are formulated separately.

97. The method or LAG-3 antagonist for use of claim 96, wherein each checkpoint inhibitor is formulated separately when the checkpoint inhibitor comprises more than one checkpoint inhibitor.

98. The method or LAG-3 antagonist for use of any one of claims 51 to 95, wherein the LAG-3 antagonist and the checkpoint inhibitor are formulated together.

99. The method or LAG-3 antagonist for use of claim 98, wherein two or more checkpoint inhibitors are formulated together when the checkpoint inhibitor comprises more than one checkpoint inhibitor.

100. The method or LAG-3 antagonist for use of claim 96 or 97, wherein the checkpoint inhibitor is administered before the LAG-3 antagonist.

101. The method or LAG-3 antagonist for use of claim 96 or 97, wherein the LAG-3 antagonist is administered before the checkpoint inhibitor.

102. The method or LAG-3 antagonist for use of any one of claims 96 to 99, wherein the LAG-3 antagonist and the checkpoint inhibitor are administered concurrently.

103. The method or LAG-3 antagonist for use of any one of claims 51 to 102, wherein the checkpoint inhibitor is administered at a flat dose.

104. The method or LAG-3 antagonist for use of any one of claims 51 to 102, wherein the checkpoint inhibitor is administered as a weight-based dose.

105. The method or LAG-3 antagonist for use of claim 103 or 104, wherein the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

106. The method of any one of claims 1 to 5 and 9 to 105 or LAG-3 antagonist for use of any one of claims 6 to 105, wherein the cancer is selected from the group consisting of breast cancer, hepatocellular cancer, gastroesophageal cancer, melanoma, bladder cancer, gastric cancer, lung cancer, kidney cancer, head and neck cancer, colon cancer, and any combination thereof.

107. The method of any one of claims 1 to 5 and 9 to 106 or LAG-3 antagonist for use of any one of claims 6 to 106, wherein the cancer is a bladder cancer.

108. The method of any one of claims 1 to 5 and 9 to 106 or LAG-3 antagonist for use of any one of claims 6 to 106, wherein the cancer is a gastric cancer.

109. The method of any one of claims 1 to 5 and 9 to 106 or LAG-3 antagonist for use of any one of claims 6 to 106, wherein the cancer is a melanoma.

110. The method of any one of claims 1 to 5 and 9 to 106 or LAG-3 antagonist for use of any one of claims 6 to 106, wherein the cancer is a lung cancer.

111. The method of any one of claims 1 to 5 and 9 to 106 or LAG-3 antagonist for use of any one of claims 6 to 106, wherein the cancer is a breast cancer.

112. The method of any one of claims 1 to 5 and 9 to 106 or LAG-3 antagonist for use of any one of claims 6 to 106, wherein the cancer is a hepatocellular cancer.

113. The method of any one of claims 1 to 5 and 9 to 112 or the LAG-3 antagonist for use of any one of claims 6 to 112, wherein the cancer is unresectable.

114. The method of any one of claims 1 to 5 and 9 to 112 or the LAG-3 antagonist for use of any one of claims 6 to 112, wherein the cancer is locally advanced.

115. The method of any one of claims 1 to 5 and 9 to 112 or LAG-3 antagonist for use of any one of claims 6 to 112, wherein the cancer is metastatic.

116. The method of any one of claims 1 to 5 and 9 to 115 or LAG-3 antagonist for use of any one of claims 6 to 115, wherein the administering treats the cancer.

117. The method of any one of claims 1 to 5 and 9 to 116 or LAG-3 antagonist for use of any one of claims 6 to 116, wherein the administering reduces the size of a tumor associated with the cancer.

118. The method of any one of claims 1 to 5 and 9 to 117 or LAG-3 antagonist for use of any one of claims 6 to 117, wherein the size of the tumor is reduced by at least about 10%, about 20%, about 30%, about 40%, or about 50% compared to the tumor size prior to the administration.

119. The method of any one of claims 1 to 5 and 9 to 118 or LAG-3 antagonist for use of any one of claims 6 to 118, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

120. The method of any one of claims 1 to 5 and 9 to 119 or the LAG-3 antagonist for use of any one of claims 6 to 119, wherein the subject exhibits stable disease after the administration.

121. The method of any one of claims 1 to 5 and 9 to 119 or the LAG-3 antagonist for use of any one of claims 6 to 119, wherein the subject exhibits a partial response after the administration.

122. The method of any one of claims 1 to 5 and 9 to 119 or the LAG-3 antagonist for use of any one of claims 6 to 119, wherein the subject exhibits a complete response after the administration.

123. A kit for treating a subject afflicted with a tumor, the kit comprising:
(a) a dosage of the LAG-3 antagonist; and
(b) instructions for using the LAG-3 antagonist in the method of any of claims 1 to 5 and 9 to 122 or the LAG-3 antagonist for use of any one of claims 6 to 122.

124. The kit of claim 123, further comprising a dosage a PD-1 pathway inhibitor.
